(19) United States
(12) Patent Application Publication
(10) Pub. No.: US 2003/0212455 A1
(43) Pub. Date: Nov. 13, 2003

(54) Identification of in vivo dna binding loci of chromatin proteins using a tethered nucleotide modification enzyme

(76) Inventors: Bas Van Steensel, Amsterdam (NL); Steven Henikoff, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/221,865

(22) PCT Filed: Mar. 16, 2001
(86) PCT No.: PCT/US01/08590

(51) Int. Cl. A61F 2/16; C12Q 1/68; A61F 2/00

(52) U.S. Cl. 623/6.6; 424/427; 435/6

(57) ABSTRACT

A novel technique is provided, designated DamID, for the identification of DNA loci that interact in vivo with specific nuclear proteins in eukaryotes. By tethering a DNA modification enzyme, in particular, E. coli DNA adenine methyl transferase (Dam), to a chromatin protein. The DNA modification enzyme (Dam) can be targeted in vivo to the native binding loci of the protein, resulting in local DNA modification. Sites of DNA modification can subsequently be mapped using modification-specific restriction enzymes, antibodies, or DNA array methods. DNA Modification Identification (DamID) has potential for genome-wide mapping of in vivo target binding sites of chromatin proteins in various eukaryotes.

### RELATED APPLICATIONS

This application is a continuation in part of U.S. patent application Ser. No. 60/______ , filed Mar. 1, 2001, and a continuation in part of U.S. patent application Ser. No. 60/190,362, filed Mar. 16, 2000, the disclosures of which are incorporated herein by reference in their entirety.

### BACKGROUND OF THE INVENTION

Chromatin is the highly complex structure consisting of DNA and hundreds of directly and indirectly associated proteins. Most chromatin proteins exert their regulatory and structural functions by binding to specific chromosomal loci. Knowledge of the nature of the in vivo target loci is essential for the understanding of the functions and mechanisms of action of chromatin proteins. Interactions between protein complexes and DNA are at the heart of essential cellular processes such as transcription, DNA replication, chromosome segregation, and genome maintenance. High-resolution, genome-wide maps of binding sites of these proteins can provide a valuable resource for researchers studying chromosome organization, chromatin structure, and gene regulation, but such comprehensive maps are currently unavailable. Therefore, techniques are needed to identity DNA loci that interact in vivo with specific proteins.

At present, only a few techniques are available to localize the genomic loci recognized by DNA binding proteins (reviewed in Simpson, Curr. Opin. Genet. Dev. 9:225-229 (1999)). In situ cross-linking methods followed by the immunoprecipitation purification of protein-DNA complexes have been used to test the interaction of individual chromosomal loci with a particular chromatin protein (Solomon et al., Cell 53:937-947 (1988); Law et al., Nucleic Acids Res. 26:919-924 (1988); Orlando et al., Methods 11:205-214 (1997); Kuo and Allis, Methods 19:425-433 (1999); Blat et al., Cell 98:249-259 (1999); Orlando, Trends Biochem. Sci. 25:99-104 (2000)). These previously disclosed techniques have the inherent risk of artifacts induced by the cross-linking reagent, and highly specific antibodies against each protein of interest are required, as well as relatively large numbers of cells. A modification of this approach was recently employed to identify binding sites of cohesins along a complete chromosome in yeast (Blat and Kleckner, Cell 98:249-259 (2000).

Another method employs in vivo targeting of a nuclease to mark binding sites of a specific protein (Lee et al., Proc. Natl. Acad. Sci. USA 95:969-974 (1998)). This method has the disadvantage that the introduction of DNA breaks is likely to cause major changes in chromatin structure and activation of DNA-damage checkpoint pathways. Systemic large-scale mapping of in vivo protein binding sites in higher eukaryotes has not been reported, presumably because of technical difficulties due to the higher genome complexity and the large number of cells required for detection by, for example, immunoprecipitation.

What is needed in the art is a rapid and efficient large-scale method for the mapping of sites of protein interaction and/or protein binding sites within the genome. Production of such maps of target loci of chromatin proteins can provide new insights into chromatin structure and gene regulation in cells, tissues and entire multicellular organisms.

### SUMMARY OF THE INVENTION

The present invention provides methods and compositions for identifying the binding loci of chromatin proteins using a tethered nucleotide modification enzyme. In particular, the nucleotide modification enzyme is tethered to the chromatin protein as a fusion protein. The fusion protein can also include a peptide linker sequence between the chromatin protein and the nucleotide modification enzyme. The fusion protein can also comprise a fragment, derivative or analog of the chromatin protein which can bind specifically to the chromatin site recognized by the wild type chromatin protein. Still further the fusion protein can comprise fragments, derivatives or analogs of the nucleotide modification enzyme which retain the enzymatic activity of the native enzyme.

In the methods of the present invention a cell or population of cells is transfected with an expression vector which comprises a polynucleotide which encodes a low efficiency promoter operatively associated with a polynucleotide which encodes the chromatin protein and the nucleotide modification enzyme. The vector can further encode a peptide linker operatively associated between the chromatin protein and the nucleotide modification enzyme. In a particular embodiment of the present invention the linker sequence is the myc epitope tag.

Nucleotide modification enzymes useful in the present invention include adenine methyltransferase, cytosine methyltransferase, thymidine hydroxylase, hydroxymethyluracil β glucosyl transferase, and adenosine deaminase. In a particular embodiment of the present invention a polynucleotide encoding Escherichia coli DNA adenine methyltransferase was used as the tethered nucleotide modification enzyme.

Once the nucleotide modification enzyme has been directed to the chromatin binding site by the chromatin protein, the nucleotide modification enzyme can modify nucleotides of the chromatin in the region of the binding site. These modifications of the nucleotides can be detected by various methods including immunochemistry, Southern blot, PCR, and various types of macro- and micro-arrays. The binding loci of the chromatin protein can be identified by determining the location of the detected nucleotide modifications within the chromatin. In a specific embodiment, the loci or the chromatin proteins heterochromatin binding protein 1, GAGA factor and Drosophila DmSir2-1 gene was determined by immunocytochemistry.

The methods of the present invention also provide methods for large scale mapping of loci of chromatin proteins. The methods can be used to obtain detailed genome-wide maps of the binding patterns of chromatin proteins in, for example, cell populations grown in culture, tissues, or in cells isolated from an entire multicellular organism. The chromatin profiles can provide information into the functions and mechanisms of action of chromatin proteins on an individual cellular basis, at the tissue level, and the organism level. In a particular embodiment pairwise comparison of profiles of different chromatin proteins in the same cell type can be used to determine functional interactions (or lack thereof) between these proteins. At the level of an organism, the profiles can be used to compare the profiles between different organisms or between different states (e.g. developmental stages) of an organism.

The present invention further provides methods for producing a profile of chromatin protein loci for a cell population of interest which method comprises; transfecting the cell population with a plurality of expression vectors capable of expressing a plurality of different chromatin protein-nucleotide modification enzyme fusion proteins, each expression vector comprising a nucleic acid encoding a low efficiency promoter operatively associated with a nucleic acid encoding the different chromatin proteins and a nucleic acid encoding a nucleotide modification enzyme; culturing the transfected cells for a period of time sufficient for expression of and binding of each of the plurality of chromatin protein-nucleotide modification enzyme fusion polypeptides; and detecting the loci for each of the nucleotide modifications within the chromatin of the cell population. The profile of chromatin protein loci for the cell population is determined from the location of the DNA modifications.

The present invention also encompasses methods for comparing the profile determined for one chromatin protein to a profile determined for the same chromatin protein, or a different chromatin protein, after the cell population has been treated with an agent. Still further, the present invention encompasses methods for comparing profiles determined for a chromatin protein at, for example, different developmental stages, or for cell populations from different tissues, different organisms, or to compare differences between the binding site profiles between normal and malignant cells.

The information provided by use of the methods of the present invention can be used for diagnostic or prognostic methods, for disease or predisposition for disease. Still further, the methods can be used for various other methods for screening for agents which can effect a disease state or modulate the development and differentiation of a cell population.

### BRIEF DESCRIPTION OF THE DRAWINGS

FIG. 1A through FIG. 1D depict targeting of Dam to a specific DNA sequence. FIG. 1A depicts a schematic of Dam and GALDam fusion protein constructs. FIG. 1B indicates the position of the probed GATCs at the EP(2)0750 insertion site. The EP element is indicated by the white bar; genomic DNA by a single line. Numbers indicate distances (in base pairs (bp)) from the UAS14 array. FIG. 1C provides methylation frequencies of GATCs at indicated distances from the UAS14 array. Numbers above error bars indicate the number of individual flies tested. FIG. ID presents the ratios of methylation frequencies in GALDam1 and Me4 flies calculated from the data in FIG. 1C. Dotted line, hypothetical exponential curve fitted to the data (r=0.89). Error bars in FIG. 1C and FIG. 1D represent standard deviations.

FIG. 2 provides a schematic of Dam-HP1 and Dam-myc fusion protein constructs.

FIG. 3A through FIG. 3C depicts HP-1-targeted methylation of specific genomic loci. FIG. 3A provides an overview of genomic locations of the probes used in the present study. White areas represent euchromatin, black areas heterochromatin; hatched areas are euchromatic regions that are decorated with HP1 in polytene chromosomes 18. Black boxes mark heterochromatic loci, open boxes euchromatic loci, boxes mark loci of which HP1-association was difficult to predict beforehand (see infra). FIG. 3B demonstrates the methylation frequencies (calculated as % DpnI-released fragment) of various loci after transfection with Dam-myc (open bars) or Dam-HP1 (black bars). FIG. 3C provides ratios of methylation in Dam-HP1 transfected cells and Dam-myc transfected cells, calculated from the data in FIG. 3B. Shading is the same as the boxes in FIG. 3A. Bullets indicate ratios that are significantly different (one, p<0.05; two, p<0.01; three, p<0.001 according to the Mann-Whitney U-test) from the pooled ratios of the four heterochromatic loci (black bullets) or the five euchromatic loci (white bullets). Error bars represent standard deviations. The number of observations is indicated in parentheses.

FIG. 4A and FIG. 4B depict mapping of HP1 target loci. FIG. 4A demonstrates a chromosomal map of Cy3:Cy5 ratios (representative experiment). Probed loci are indicated by their approximate position on the cytogenetic map. Centromeres are indicated by ovals. The large heterochromatic proximal region of the X chromosome is depicted as a rectangle to the left of the centromere (not to scale). Some genes with relatively high levels of HP1 binding are labeled. FIG. 4B depicts dispersed repetitive elements (mostly transposons).

FIG. 5A through FIG. 5C depict the mapping of GAF target loci. FIG. 5A provides a chromosomal map of Cy3:Cy5 ratios (average of two experiments) using a GAF-Dam fusion protein. Some genes with relatively high levels of GAF binding are labeled. FIG. 5B depicts dispersed repetitive elements (mostly transposons). FIG. 5C depicts a box plot showing the relative abundances of GAGAG (SEQ ID NO: 4) and GAGAGAG (SEQ ID NO: 5) sequence elements in probed regions with low (open boxes) and high (filled boxes) levels of GAF binding. Horizontal lines represent the 10th, 25th, 50th (median), 75th and 90th percentiles. p-values are according to the Mann-Whitney U-test.

FIG. 6A and FIG. 6B depict the. mapping of DmSir2-1 target loci. FIG. 6A provides a chromosomal map of Cy3:Cy5 ratios (average of two experiments) for chromosomes 2, 3 and 4, and the X chromosome. FIG. 6B depicts dispersed repetitive elements (mostly transposons). Some genes of particular interest or with high levels of DmSir2-l are labeled.

### DESCRIPTION OF THE SPECIFIC EMBODIMENTS

The terms “polynucleotide” and “nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 10⁹ nucleotides or larger. Polynucleotides and nucleic acids include RNA, cDNA, genomic DNA. In particular, the polynucleotides and nucleic acids of the present invention refer to polynucleotides encoding a chromatin protein, a nucleotide modifying enzyme and/or fusion polypeptides of a chromatin protein and a nucleotide modifying enzyme, including mRNAs, DNAs, cDNAs, genomic DNA, and polynucleotides encoding fragments, derivatives and analogs thereof. Useful fragments and derivatives include those based on all possible codon choices for the same amino acid, and codon choices based on conservative amino acid substitutions. Useful derivatives further include those having at least 50% or at least 70% polynucleotide sequence identity, and more preferably 80%, still more preferably 90% sequence identity, to a native chromatin binding protein or to a nucleotide modifying enzyme.

The term “oligonucleotide” refers to a polynucleotide of from about six (6) to about one hundred (100) nucleotides or more in length. Thus, oligonucleotides are a subset of polynucleotides. Oligonucleotides can be synthesized manually, or on an automated oligonucleotide synthesizer (for example, those manufactured by Applied BioSystems (Foster City, Calif.)) according to specifications provided by the manufacturer or they can be the result of restriction enzyme digestion and fractionation.

The term “primer” as used herein refers to a polynucleotide, typically an oligonucleotide, whether occurring naturally, as in an enzyme digest, or whether produced synthetically, which acts as a point of initiation of polynucleotide synthesis when used under conditions in which a primer extension product is synthesized. A primer can be single-stranded or double-stranded.

The term “nucleic acid array” as used herein refers to a regular organization or grouping of nucleic acids of different sequences immobilized on a solid phase support at known locations. The nucleic acid can be an oligonucleotide, a polynucleotide, DNA, or RNA. The solid phase support can be silica, a polymeric material, glass, beads, chips, slides, or a membrane. The methods of the present invention are useful with both macro- and micro-arrays.

The term “chromatin” as used herein refers to a complex of DNA and protein, both in vitro and in vivo. This includes all proteins that are directly contacting DNA, and also proteins that are part of a protein or ribonucleoprotein complex that may be associated with DNA. A chromatin protein may or may not directly contact DNA. Chromatin also includes proteins that are transiently associated with DNA, with DNA-protein, or with DNA-ribonucleoprotein complexes, i.e., only during a part of the cell cycle.

“Chromatin protein” includes, but is not limited to histones, transcriptional factors, centromere proteins, heterochromatin proteins, euchromatin proteins, condensins, cohesins, origin recognition complexes, histone kinases, dephosphorylases, acetyltransferases, deacetylases, methyltransferases, demethylases, and other enzymes that covalently modify histone, DNA repair proteins, proteins involved in DNA replication, proteins involved in transcription, proteins part of dosage compensation complexes and X-chromosome inactivation, proteins that are part of chromatin remodeling complexes, telomeric proteins, and the like.

“Chromatin protein-enzyme fusion polypeptide” refers to a polypeptide encoded by a polynucleotide encoding the chromatin protein operatively associated with a polynucleotide which encodes a nucleotide modification enzyme. Also encompassed within this definition are polynucleotides which encode a functionally active fragment, derivative or analog of the chromatin protein or nucleotide modification enzyme. The term “polypeptide” refers to a polymer of amino acids and its equivalent and does not refer to a specific length of the product; thus, peptides, oligopeptides and proteins are included within the definition of a polypeptide. A “fragment” refers to a portion of a polypeptide having typically at least 10 contiguous amino acids, more typically at least 20, still more typically at least 50 contiguous amino acids of the chromatin protein. A “derivative” is a polypeptide which is identical or shares a defined percent identity with the wild-type chromatin protein or nucleotide modification enzyme. The derivative can have conservative amino acid substitutions, as compared with another sequence. Derivatives further include, for example, glycosylations, acetylations, phosphorylations, and the like. Further included within the definition of “polypeptide” are, for example, polypeptides containing one or more analogs of an amino acid (e.g., unnatural amino acids, and the like), polypeptides with substituted linkages as well as other modifications known in the art, both naturally and non-naturally occurring. Ordinarily, such polypeptides will be at least about 50% identical to the native chromatin binding protein or nucleotide modification enzyme acid sequence, typically in excess of about 90%, and more typically at least about 95% identical. The polypeptide can also be substantially identical as long as the fragment, derivative or analog displays similar functional activity and specificity as the wild-type chromatin protein or nucleotide modification enzyme.

The terms “amino acid” or “amino acid residue”, as used herein, refer to naturally occurring L amino acids or to D amino acids as described further below. The commonly used one- and three-letter abbreviations for amino acids are used herein (see, e.g., Alberts et al., Molecular Biology of the Cell, Garland Publishing, Inc., New York (3d ed. 1994)).

The term “isolated” refers to a nucleic acid or polypeptide that has been removed from its natural cellular environment. An isolated nucleic acid is typically at least partially purified from other cellular nucleic acids, polypeptides and other constituents.

“Functionally active polypeptide” refers to those fragments, derivatives and analogs displaying the functional activities associated with a fall length chromatin protein or nucleotide modifying enzyme (e.g., binding the chromatin protein locus in the case of the fragments, derivatives of the chromatin protein and those fragments, derivatives and analogs of the nucleotide modifying enzyme which are capable of modifying a nucleotide in the case of the nucleotide modification enzyme, and the like).

The terms “identical” or “percent identity,” in the context of two or more nucleic acids or polypeptide sequences, refer to two or more sequences or subsequences that are the same or have a specified percentage of nucleotides or amino acid residues that are the same, when compared and aligned for maximum correspondence, as measured using one of the following sequence comparison algorithms, or by visual inspection.

The phrase “substantially identical,” in the context of two nucleic acids or polypeptides, refers to two or more sequences or subsequences that have at least 60%, typically 80%, most typically 90-95% nucleotide or amino acid residue identity, when compared and aligned for maximum correspondence, as measured using one of the following sequence comparison algorithms, or by visual inspection. An indication that two polypeptide sequences are “substantially identical” is that one polypeptide is immunologically reactive with antibodies raised against the second polypeptide.

“Similarity” or “percent similarity” in the context of two or polypeptide sequences, refer to two or more sequences or subsequences that are the same or have a specified percentage of amino acid residues or conservative substitutions thereof, that are the same, when compared and aligned for maximum correspondence, as measured using one of the following sequence comparison algorithms, or by visual inspection. By way of example, a first amino acid sequence can be considered similar to a second amino acid sequence when the first amino acid sequence is at least 30%, 40%, 50%, 60%, 70%, 75%, 80%, 90%, or even 95% identical, or conservatively substituted, to the second amino acid sequence when compared to an equal number of amino acids as the number contained in the first sequence, or when compared to an alignment of polypeptides that has been aligned by a computer similarity program known in the art, as discussed below.

The term “substantial similarity” in the context of polypeptide sequences, indicates that the polypeptide comprises a sequence with at least 70% sequence identity to a reference sequence, or preferably 80%, or more preferably 85% sequence identity to the reference sequence, or most preferably 90% identity over a comparison window of about 10-20 amino acid residues. In the context of amino acid sequences, “substantial similarity” further includes conservative substitutions of amino acids. Thus, a polypeptide is substantially similar to a second polypeptide, for example, where the two peptides differ only by one or more conservative substitutions.

The term “conservative substitution,” when describing a polypeptide, refers to a change in the amino acid composition of the polypeptide that does not substantially alter the polypeptide's activity. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar, and the like) such that the substitution of even critical amino acids does not substantially alter activity. Conservative substitution tables providing functionally similar amino acids are well known in the art. For example, the following six groups each contain amino acids that are conservative substitutions for one another: 1) alanine (A), serine (S), threonine (T); 2) aspartic acid (D), glutamic acid (E); 3) asparagine (N), glutamine (Q); 4) arginine (R), lysine (K); 5) isoleucine (I), leucine (L), methionine (M), valine (V); and 6) phenylalanine (F), tyrosine (Y), tryptophan (W). (See also Creighton, Proteins, W. H. Freeman and Company (1984).) In addition, individual substitutions, deletions or additions that alter, add or delete a single amino acid or a small percentage of amino acids in an encoded sequence are also “conservative substitutions.”

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith & Waterman (Adv. Appl. Math. 2:482 (1981), which is incorporated by reference herein), by the homology alignment algorithm of Needleman & Wunsch (J. Mol. Biol. 48:443-53 (1970), which is incorporated by reference herein), by the search for similarity method of Pearson & Lipman (Proc. Natl. Acad. Sci. USA 85:2444-48 (1988), which is incorporated by reference herein), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al. (eds.), Current Protocols in Molecular Biology, John Wiley and Sons, New York (1996)).

One example of a useful algorithm is PILEUP. PILEUP creates a multiple sequence alignment from a group of related sequences using progressive, pairwise alignments to show the percent sequence identity. It also plots a tree or dendrogram showing the clustering relationships used to create the alignment. PILEUP uses a simplification of the progressive alignment method of Feng and Doolittle (J. Mol. Evol. 25:351-60 (1987), which is incorporated by reference herein). The method used is similar to the method described by Higgins & Sharp (Comput. Appl. Biosci. 5:151-53 (1989), which is incorporated by reference herein). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described by Altschul et al. (J. Mol. Biol. 215:403-410 (1990), which is incorporated by reference herein). (See also Zhang et al.,Nucleic Acid Res. 26:3986-90 (1998); Altschul et al., Nucleic Acid Res. 25:3389-402 (1997), which are incorporated by reference herein). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990), supra). These initial neighborhood word hits act as seeds for initiating searches to find longer HSPs containing them. The word hits are then extended in both directions along each sequence for as far as the cumulative alignment score can be increased. Extension of the word hits in each direction is halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff & Henikoff, Proc. Natl. Acad. Sci. USA 89:10915-9 (1992), which is incorporated by reference herein) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g. Karlin & Altschul, Proc. Natl. Acad. Sci. USA 90:5873-77 (1993), which is incorporated by reference herein). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more typically less than about 0.01, and most typically less than about 0.001. Further, a polypeptide is typically substantially identical to a second polypeptide, for example, where the two peptides differ only by conservative substitutions.

The terms “transformation” or “transfection” means a process of stably or transiently altering the genotype of a recipient cell or microorganism by the introduction of polynucleotides. This is typically detected by a change in the phenotype of the recipient cell or organism. The term “transformation” is generally applied to microorganisms, while “transfection” is used to describe this process in cells derived from multicellular organisms.

Generally, other nomenclature used herein and many of the laboratory procedures in cell culture, molecular genetics and nucleic acid chemistry and hybridization, which are described below, are those well known and commonly employed in the art. (See generally Ausubel et al. (1996) supra; Sambrook et al., Molecular Cloning: A Laboratory Manual, Second Edition, Cold Spring Harbor Laboratory Press, New York (1989), which are incorporated by reference herein). Standard techniques are used for recombinant nucleic acid methods, polynucleotide synthesis, preparation of biological samples, preparation of cDNA fragments, isolation of mRNA and the like. Generally enzymatic reactions and purification steps are performed according to the manufacturers' specifications.

The present invention provides methods and compositions for use in identifying the in vivo target loci of chromatin proteins in a living cell or in populations of living cells including, for example, specific tissues or cell populations isolated from an entire multicellular organism. More specifically, the methods and compositions comprise the use of the chromatin protein, or chromatin binding proteins, and chromatin binding fragments or derivatives thereof, linked or fused to an enzyme which modifies at least one, and typically more than one, nucleotide in the region associated with the target loci. In one specific example the modification enzyme is DNA adenine methyl transferase (Dam). Nucleotide sequences which have been modified are identified using, for example, an antibody specific for the modified nucleotide, restriction enzymes specific for particular modified nucleotide sequences, or by DNA micro-array methods. The technique, designated herein DamID (for DNA adenine methyl transferase IDentification), is sensitive and specific, and does not have any of the disadvantages of prior methods.

Chromatin, as above, is a complex of DNA and protein, e.g., in the nucleus of a cell in interphase. Many of these interactions require the presence of chromatin proteins which exert their regulatory and structural functions by binding to, or complexing with other proteins or nucleic acids, with a specific chromosomal loci. In the present invention the chromatin protein, or a specific binding fragment or derivative thereof is used to direct a nucleotide modification enzyme to the specific loci recognized by the chromatin protein. Any chromatin protein, or protein which recognizes a specific loci or sequence of nucleotides can be used to produce the fusion protein of the present invention. In specific embodiments of the present invention nucleotide sequences encoding Heterochromatin protein 1 (HP1), which binds predominantly to pericentric genes and transposable elements, GAGA factor (GF) which associates with euchromatic genes that are enriched in (GA)ₙ motifs, and a Drosophila homolog of the yeast Sir2 gene (DmSir2-1) which associates with certain active genes were used to construct exemplary fusion proteins of the invention.

A specific binding fragment or derivative of a chromatin protein comprises that portion of the chromatin protein or protein-nucleic acid complex required to recognize and bind the chromosomal loci or region recognized by the native chromatin protein. For example, a specific binding fragment of a Heterochromatin protein 1 (HP1), which binds predominantly to pericentric genes and transposable elements, GAGA factor (GF) which associates with euchromatic genes that are enriched in (GA)ₙ motifs, or a Drosophila homolog of the yeast Sir2 gene (DmSir2-1) which associates with certain active genes can be used to construct a fusion protein of the invention. Fragments, derivatives or analogs of a chromatin protein or protein complex can be tested for the desired activity by procedures known in the art, including but not limited to the functional assays to determine whether the fragment recognizes and binds the target loci or nucleotide sequence recognized by the native full length chromatin binding protein. The affinity or avidity of the binding to the target loci or nucleotide sequence can be the same, less or greater than the affinity or avidity of the native full length protein. It is only necessary that the fragment, derivative or analog recognize and bind the target loci or sequence. In addition, the chromatin polypeptide fragment, derivative, or analog can be tested for the desired activity in the fusion protein to ensure localization to the appropriate loci.

Polypeptide derivatives include naturally-occurring amino acid sequence variants as well as those altered by substitution, addition or deletion of one or more amino acid residues that provide for functionally active molecules. Polypeptide derivatives include, but are not limited to, those containing as a primary amino acid sequence all or part of the amino acid sequence of a native chromatin polypeptide including altered sequences in which one or more functionally equivalent amino acid residues (e.g., a conservative substitution) are substituted for residues within the sequence, resulting in a silent change.

In another aspect, polypeptides of the present invention include those peptides having one or more consensus amino acid sequences shared by all members of the chromatin protein family members, but not found in other proteins. Database analysis indicates that these consensus sequences are not found in other polypeptides, and therefore this evolutionary conservation reflects the nucleotide target binding-specific function of chromatin polypeptides. Chromatin polypeptide family members, including fragments, derivatives and/or analogs comprising one or more of these consensus sequences, are also within the scope of the invention.

In another aspect, a polypeptide consisting of or comprising a fragment of a chromatin polypeptide having at least 5 contiguous amino acids of the chromatin polypeptide which recognize the specific target nucleotide sequence is provided. In other embodiments, the fragment consists of at least 20 or 50 contiguous amino acids of the chromatin polypeptide. In a specific embodiment, the fragments are not larger than 35, 100 or even 200 amino acids.

Fragments, derivatives or analogs of chromatin polypeptide include, but are not limited to, those molecules comprising regions that are substantially similar to a chromatin polypeptide or fragments thereof (e.g., in various embodiments, at least 30%, 40%, 50%, 60%, 70%, 75%, 80%, 90%, or even 95% identity or similarity over an amino acid sequence of identical size), or when compared to an aligned sequence in which the alignment is done by a computer sequence comparison/alignment program known in the art, as described above, or whose coding nucleic acid is capable of hybridizing to a nucleic acid sequence encoding a chromatin protein, under high stringency, moderate stringency, or low stringency conditions.

The choice of hybridization conditions will generally be guided by the purpose of the hybridization, the type of hybridization (DNA-DNA or DNA-RNA), and the level of relatedness between the sequences. Methods for hybridization are well established in the literature; See, for example: Sambrook, supra.; Hames and Higgins, eds, Nucleic Acid Hybridization A Practical Approach, IRL Press, Washington D.C., (1985); Berger and Kimmel, eds, Methods in Enzymology, Vol. 52, Guide to Molecular Cloning Techniques, Academic Press Inc., New York, N.Y., (1987); and Bothwell et al., eds, Methods for Cloning and Analysis of Eukaryotic Genes, Jones and Bartlett Publishers, Boston, Mass. (1990); which are incorporated by reference herein in their entirety. The stability of nucleic acid duplexes will decrease with an increased number and location of mismatched bases; thus, the stringency of hybridization may be used to maximize or minimize the stability of such duplexes. Hybridization stringency can be altered by: adjusting the temperature of hybridization; adjusting the percentage of helix-destabilizing agents, such as formamide, in the hybridization mix; and adjusting the temperature and salt concentration of the wash solutions. In general, the stringency of hybridization is adjusted during the post-hybridization washes by varying the salt concentration and/or the temperature. Stringency of hybridization may be reduced by reducing the percentage of formamide in the hybridization solution or by decreasing the temperature of the wash solution. High stringency conditions involve high temperature hybridization (e.g., 65-68° C. in aqueous solution containing 4 to 6× SSC, or 42° C. in 50% formamide) combined with washes at high temperature (e.g., 5 to 25° C. below the Tₘ) at a low salt concentration (e.g., 0.1× SSC). Reduced stringency conditions involve lower hybridization temperatures (e.g., 35-42° C. in 20-50% formamide) with washes at intermediate temperature (e.g., 40 to 60° C.) and in a higher salt concentration (e.g., 2 to 6× SSC). Moderate stringency conditions involve hybridization at a temperature between 50° C. and 55° C. and washes in 0.1× SSC, 0.1% SDS at between 50° C. and 55° C.

Nucleotide modifying enzymes, fragments, derivatives and analogs thereof useful in the present invention are those which can modify one or more nucleotides in a nucleic acid sequence, such as an RNA, DNA, or the like, under conditions found in a live cell and in a manner which is detectable. The enzyme must also modify the nucleotides in a manner which is not toxic to the cell. In other words, the cell or organism must be able to continue to proliferate and differentiate in a normal manner. For the modification to be detectable, an enzyme is selected which modifies the nucleotide in a manner which is not typical of a modification commonly found in the cell being assayed. For instance, in eukaryotic cells it is typical to select as the modification enzyme, for example, DNA adenine methyl transferase because methylation of adenine is not common in eukaryotic cells. Additional nucleotide modification enzymes useful in the present invention include, for example, but are not limited to, adenine methyltransferases, cytosine methyltransferases, thymidine hydroxylases, hydroxymethyluracil β-glucosyl transferases, adenosine deaminases, and the like. However, as described in more detail below, within one embodiment, a modification of the method of the present invention relies on an endogenous modification enzyme to modify DNA in a cell, the sites of such modifications are then determined by a variety of detection means, including the use of nucleic acid arrays.

In the methods of the present invention, the DNA modification enzyme, fragment, derivative, or analog thereof, is targeted to the loci associated with the binding of the chromatin protein by the chromatin protein, fragment, derivative or analog thereof, as a fusion protein. Typically, the polypeptides which comprise the chromatin protein and the DNA modification enzyme are separated from one another by one or more amino acid residues which comprise a linker sequence. The linker can be from about 1 to about 1000 amino acid residues, or more. Typically, the linker sequence is from about 3 to about 300 amino acid residues. The amino acid sequence can be from another polypeptide or can be an artificial sequence of amino acid residues, such as, for example, Gly and Ser residues which provide a flexible linear amino acid sequence allowing the amino acid sequences for the chromatin polypeptide and the nucleotide modification enzyme to fold into an active configuration. In a particular embodiment of the present invention a linker peptide comprising the myc-epitope tag GluGlnLysIleSerGluGluAspLeu (SEQ ED NO: 1) was inserted between the chromatin polypeptide and the nucleotide modification enzyme DNA adenine methyl transferase.

Expression of the Chromatin Protein-Nucleotide Modification Enzyme Fusion Protein:

The nucleotide sequence coding for a chromatin polypeptide-nucleotide modification enzyme fusion protein, or a functionally active derivative, analog or fragment thereof, can be inserted into an appropriate expression vector (i.e., a vector which contains the necessary elements for the transcription and translation of the inserted polypeptide-coding sequence). The necessary transcriptional and translational signals can also be supplied by a native gene and/or its flanking regions. A variety of vector systems can be utilized to express the polypeptide fusion-coding sequence. The choice of vector will be dependent on the cell to be transfected. The expression elements of vectors vary in their strengths and specificities. Depending on the cell-vector system utilized, any one of a number of suitable transcription and translation elements can be used. In specific embodiments, fusion proteins of the HP1, GAF and DmSir2-1 chromatin proteins fused with the nucleotide modification enzyme, E. coli DNA adenine methyl transferase, genes are expressed, or a nucleic acid sequence encoding a functionally active portion of the fusion proteins are expressed in, for example, Drosophila cells.

Any of the methods previously described for the insertion of DNA fragments into a vector can be used to construct expression vectors containing a chimeric gene consisting of appropriate transcriptional/translational control signals and the polypeptide coding sequences. These methods include in vitro recombinant DNA and synthetic techniques and in vivo recombinants (genetic recombination). Expression of a nucleic acid sequence encoding a fusion protein of the present invention or a fragment thereof can be regulated by a second nucleic acid sequence so that the fusion polypeptide or specific binding fragment is expressed in a host transformed with the recombinant DNA molecule. For example, expression of a fusion polypeptide can be controlled by any promoter/enhancer element known in the art. Promoters typically used in the present invention are those which provide low levels of expression of the fusion protein. Low levels of expression of the fusion protein are desired to avoid high background modification of non-targeted sequences. Suitable promoters can be selected empirically for each fusion protein by routine methods well known to the skilled artisan. Promoters suitable for use in the present invention include, but are not limited to, most heat shock promoters, for example, the hsp70 promoter, and various modified promoters, such as a truncated CMV promoter, and the like.

The chromatin protein-nucleotide modification enzyme fusion protein when expressed migrates to the loci or binding site recognized by the chromatin protein. Once bound, the nucleotide modification enzyme modifies the appropriate nucleotides within a distance of the loci recognized by the chromatin protein. It is important that the modification of a sufficient number of nucleotide residues to provide a detectable signal is not toxic to the cells, tissues or organism being tested. Therefore, as above, promoters which provide for low levels of expression are used and nucleotide modification enzymes which provide non-toxic nucleotide modifications are used.

Detection of Chromatin Binding Sites:

Several methods are available for the detection of modified nucleotides in the vicinity of the binding loci recognized by the chromatin protein. These include, but are not limited to, immunohistochemistry, Southern blot analysis, PCR analysis and array (i.e., macro- and micro-array) analysis.

In a typical embodiment, cells are grown or collected on a solid phase appropriate for microscopy. For example, transformed cells can be cultured on a glass microscope cover slip. The cells are then fixed and washed. An antibody specific for the nucleotide modification carried out by the nucleotide modification enzyme of the fusion protein is added. The antibody can be either polyclonal antisera or a monoclonal antibody. Antibody can be labeled directly or a second labeled antibody can be used to detect the nucleotide modification. Following an incubation period the cells are washed and the antibody is detected providing a location within the nucleus where the chromatin protein complexes within the chromatin. In one particular embodiment the cells are prepared as mitotic spreads by methods well known to the skilled artisan.

A wide variety of labels can be employed for detection of the nucleotide modification. For example, the label can be, chemiluminescent, enzyme, fluorophor, or a radioactive moiety, and the like. Typically, fluorescent labels, such as, fluorescein, phycoerythrin (PE), Cy3, Cy5, Cy7, Texas Red, allophycocyanin (APC), Cy7APC, Cascade Blue, Cascade Yellow, and the like, can be used. Methods for labeling antibodies are well known to the skilled artisan.

In still another embodiment Southern blot can be used to map the region of the chromatin where a nucleotide modification has occurred. Typically, genomic DNA is isolated from a population of cells transformed with the vector capable of expressing the chromatin binding protein-nucleotide modification enzyme fusion polypeptide by methods well known to the skilled artisan. The population of cells useful in the methods of the present invention can be isolated from cells grown in vitro, isolated from a single tissue, or isolated from a multicellular organism.

The isolated DNA is digested with a restriction enzyme specific for the enzyme modification. In one embodiment of the present invention DpnI, which recognizes the nucleotide sequence G^{m6}A↓C, or DpnII, which recognizes the nucleotide sequence GA↓TC, was used to cut the isolated genomic DNA. Other restriction enzymes and their associated methylases which can be used in the present invention are well known in the art (See, for example, Roberts and Macelis, Nuc. Acids Res. 26:338-350 (1998) incorporated herein by reference). Following digestion the DNA can be separated by size by any of a number of methods known in the art. Typically, 1.5% agarose electrophoresis is used. Detection of regions of methylation can be accomplished using labeled probes specific for a particular GATC sequence of a gene of interest.

In still another embodiment of the present invention PCR methods can be used to detect region of the chromatin recognized by a chromatin binding protein. This method comprises isolation and extraction of the genomic DNA from a cell sample. As with Southern blot the genomic DNA is digested with a restriction enzyme specific for a modified nucleotide sequence and compared to a digest with a restriction enzyme which recognizes the unmodified nucleotide sequence. Primers are selected to hybridize with nucleotide sequences either on each side of a known restriction site or on each side of a restriction site pair and the nucleotide sequence containing the restriction site(s) is amplified in the presence of labeled nucleotide residues. The levels of methylation can then be determined for each site of interest. In particular embodiments of the present invention the methylation of GATC sequences were determined by this method using the HP1-DNA adenine methyl transferase fusion protein for, but not limited to, the histone gene cluster repeat HisC), the 28S gene in the rDNA repeat, the cubitus interruptus (cis) gene and the S-adenosyl decarboxylase gene.

In another embodiment, the methods of the present invention can be used in combination with DNA microarray technology (Pease et al., Proc. Natl. Acad. Sci. USA 91:5022-5026 (1994); Schena et al., Science 270:467-470 (1995)). Genomic regions that contain modified nucleotides are purified, labeled, and used to probe a DNA array. Such an approach allows the identification of target genes of specific proteins at a genome-wide scale.

In a specific embodiment, genomic DNA is isolated from cells transfected with an expression vector which can produce a chromatin binding protein-nucleotide modification enzyme fusion protein and, as a control, cell which have not been transfected. The isolated genomic DNA is digested with a restriction enzyme specific for the nucleotide sequence containing the nucleotide modification. The digested DNA is size fractionated and fragments smaller than about 2.5 kb are typically added to a test array. Arrays useful in the present invention include, but are not limited to cDNA, DNA, DNA selected to contain primarily chromatin binding regions or protein binding regions, and the lice. Each sample of methylated and control fractionated DNA can be labeled with, for example, a different fluorescent label. The labeled samples are mixed and applied to the array under condition conducive for hybridization using methods well known in the art. The arrays are scanned for the detection of the two labels and the loci recognized by the chromatin protein can be mapped.

Additional methods for the purification of methylated DNA regions, which can be applied separately or used in various combinations in order to further increase the purity of the isolated methylated regions include the following:

Methylated DNA fragments can be affinity purified using antibodies against ^{m6}A. Monoclonal antibody (for example, clone P1A8) which specifically recognize methyl-6-adenine (^{m6}A) have been generated using a procedure previously described (Bringmann et al., FEBS Lett. 213:309-315 (1987)). The antibody obtained can be used in conjunction with the restriction endonuclease DpnI to affinity purify methylated DNA fragments. First, purified genomic DNA is digested with DpnI, which results into exposure of ^{m6}A at the blunt ends of the digestion products. Antibody was allowed to bind to the exposed ^{m6}A. Antibody-DNA complexes were then isolated using (for example) protein A—sepharose beads (Amersham) pre-coated with rabbit-anti-mouse antibody. After purification, methylated DNA fragments were eluted from the antibody by incubation with 20 mM free methyl-6-adenosine.

Further, methylation-specific PCR amplification has been used to isolate methylated DNA fragments. After digestion with DpnI, an excess of double-stranded adaptor oligonucleotide (with non-phosphorylated 5′ ends to prevent self-ligation of the oligonucleotide) were ligated to the exposed blunt DNA ends using T4 DNA ligase. Because DpnI cuts only methylated GATC sequences, the adaptor only ligated to methylated DNA ends. The ligated fragments were specifically amplified by PCR using a primer complementary to the adaptor sequence. The specificity of this procedure can be further enhanced using either of two modifications.

In one modification, prior to the DpnI digestion, genomic DNA is treated with a DNA phosphatase such as alkaline phosphatase. This prevents ligation of the adaptor to DNA ends that were not the product of DpnI digestion (e.g., DNA breaks resulting from mechanical shearing or contaminant endonuclease activity during the purification of genomic DNA). Also, prior to PCR amplification, the ligated DNA sample can be digested with DpnII, which cuts only unmethylated GATCs. Any ligation products containing unmethylated GATCs will be destroyed by this treatment; the hemi-methylated ligation junctions were found to be resistant to DpnII.

In a particular embodiment of the present invention a cDNA library comprising randomly selected ESTs from Drosophila, and a library of 140 cDNA and 20 genomic DNA fragments cloned and selected to be unique were used. PCR amplification products of the selected clones were “spotted” onto a coated solid phase (ploy L-lysine coated glass microscope slides) by methods well known to the skilled artisan. Purified methylated and fractionated DNA from a test sample and a control were labeled with Cy3- or Cy5-DTP by random priming the samples were mixed prior to adding them to the array for hybridization with yeast tRNA and unlabeled DpnI-digested plasmid DNA encoding the fusion protein. Arrays were scanned using a fluorescent scanner and the data processed to provide a ratio of Cy3:Cy5 binding.

Analysis of microarrays using methods of the present invention used cDNA probes. Thus, the analysis focused on transcribed regions. Arrays of genomic fragments systematically covering promoter and enhancer regions can provide detailed insight into the associations of chromatin proteins with cis-acting elements. The disclosed in vivo methods of the present invention demonstrate that methylation by tethered Dam spread over about 2 to about 5 kb from a discrete protein-binding sequence (Example 1), indicating that binding sites can be mapped with a resolution of a few kb. In certain cases, sequence comparison of all identified target loci can reveal common sequence elements that potentially mediate the recruitment of the chromatin protein, thus effectively increasing the mapping resolution.

Uses of DamID:

The methods of the present invention provide simple and straight forward methods for large scale mapping of target loci of chromatin proteins yielding highly reproducible results. The amount of data that can be obtained with this approach was primarily limited by the size of the DNA microarray. The method can be used to obtain extremely detailed genome-wide maps of the binding patterns of chromatin proteins, for example in cell populations grown in culture, tissues, or in cells isolated from an entire multicellular organism. Such ‘chromatin profiles’ can yield unprecedented insights into the functions and mechanisms of action of chromatin proteins on an individual cellular basis, at the tissue level, and the organism level. Furthermore, pairwise comparison of profiles of different chromatin proteins in the same cell type can reveal functional interactions (or lack thereof) between these proteins. At the level of an organism, the profiles can be used to compare the profiles between different organisms or between different states (e.g., developmental stages) of an organism. The power of the present approach is illustrated by comparative profiling of HP1 and DmSir2-1, which indicated that DmSir2-1 was not a heterochromatin protein.

Chromatin profiling can become a powerful tool in the analysis of cellular differentiation. It is anticipated that chromatin profiles made available using the methods disclosed herein for many proteins will be unique for specific cell types. Systematic mapping of such profiles can provide fundamental new insights into the mechanisms of cellular differentiation and transformation to a malignant condition. The methods as disclosed herein based on chromatin protein targeting of a nucleotide modification enzyme can be particularly useful in mammalian cells, in which other global mapping approaches based on chromatin immunoprecipitation methods (Blat and Kleckner, Cell 98:249-259 (1999)) may fail due to the high complexity of the genome and insufficient specificity of antibodies.

Moreover, in analogy to mRNA expression profiles (Golub et al. Science 286:531-537 (1999); Ross et al., Nature Genet. 24:227-235 (2000)), chromatin profiles can be used in studies of cellular pathology. One important application can be in the discovery and prediction of cancer types. Different classes of tumor cells are likely to display distinct chromatin profiles, and these profiles may therefore have high analytic and diagnostic value. The wide variety of chromatin proteins can allow a much more detailed and robust classification of cancer types than expression profiling, which relies on only one data set (i.e., mRNA abundances) per cell type.

Methods of the present invention can also be used to provide chromatin profiles of individuals with immune deficiency or auto immune conditions as well as examining chromatin changes in reaction to various drags and other agents. In addition chromatin binding profiles can be constructed for responses to various disease causing organisms and expression profiles can be constructed for any transcription factor of other regulatory molecule or agent.

In another embodiment of the invention, the described methods can be applied to obtaining a methylation profile. Within this method and unlike chromatin profiling which requires the introduction of a fusion protein, genomic DNA is obtained from a cell, tissue or organism of interest and from a control cell, tissue, or organism of interest. The genomic DNA, having been methylated by endogenous methylases, is digested by incubation with a methylation-sensitive restriction endonuclease in the same manner as described for chromatin profiling. Subsequent steps are identical to those used for chromatin profiling described above, with the exception that the comparison made is between two different cell types or genotypes or between a cell, tissue or organism and a control cell, tissue, or organism, respectively. Using methylation profiling, the resulting hybridization to an array, is dependent upon the density of cleavage of endogenous methylation sites by the methylation sensitive endonuclease. The methylation sensitive endonuclease useful in this embodiment of the present invention can be those that either cut at a methylation site or fail to cut at a particular methylation site. When an endonuclease that cuts at a methylation site is used, smaller sized fractions from, for example, sucrose gradient centrifugation or another fractionation procedure contain molecules that are hypermethylated relative to larger sized fractions. In contrast to where the endonuclease used in the method is one that fails to cut at a methylation site, the larger sized fractions contain the molecules that are hypermethylated relative to smaller sized fractions.

Within one example, the restriction enzyme HpaII fails to cut -CCGG- sites methylated on the second C, where -CG- sites are naturally methylated in many organisms, including humans. Therefore, genomic regions that are densely methylated will be protected from cleavage relative to genomic regions that are weakly methylated. Cancer cells display characteristic differences in CpG methylation patterns, and thus one application of methylation profiling is to characterize methylation differences between cancer and non-cancer cells for diagnostic purposes. In this example, genomic DNA from cancer cells that is cleaved with HpaII (or some other methylation-sensitive endonuclease) is labeled with the Cy3 dye, and that from normal cells similarly cleaved with HpaII is labeled with the Cy5 dye (or vice versa).

The samples are mixed in equal proportions and the mixture is used to probe arrays, e.g., a microarray, displaying human genomic sites. By reading out the differential hybridization as evidenced by differential detection of the label, one can compare and contrast the methylation profile of the cancer cells relative to the non-cancer cells. Methylation profiling is thus similar to chromatin profiling, except that no fusion protein is needed and different restriction endonucleases are employed.

The following examples are offered by way of illustration, not by way of limitation.

### EXAMPLE 1

In this example a chromatin protein fusion protein with E. coli DNA adenine methyl transferase linked with Heterochromatin protein 1 (HP1) was used to identify DNA loci that interact with HP1 in D. melanogaster.

Expression vectors: The Dam open reading frame was amplified by PCR from plasmid YCpGAL-EDAM (Wines et al., Chromosoma 104:332-340 (1996)) and cloned into pCaSpeR-hs followed or preceded by a linker oligonucleotide encoding the myc-epitope tag GluGlnLysIleSerGluGluAspLeu (SEQ ID NO: 1). Resulting in vectors pNDamMyc and pCMycDam, respectively. Vector pNDamMyc carries a stop codon 15 amino acid residues after the myc-tag, and was used to express the Dam-myc protein. A fragment encoding amino acid residues 1-145 of GAL4 was amplified by PCR from plasmid pSPGAL1-145 (provided by S. M. Parkhurst, Fred Hutchinson Cancer Research Center, Seattle, Wash.) and cloned in-frame into vector pCMycDam, resulting in plasmid pGALDam. The full-length ORF of D. melanogaster HP1 was amplified by PCR from plasmid pTH5 (Eissenberg et al., Proc. Natl. Acad. Sci. USA 87:9923-9927 (1990), incorporated herein by reference) and cloned in-frame into pNDamMyc, resulting in plasmid pDamHP1.

Cell culture and immunocytochemistry: Kc167 cell culture and transfections were performed as described (Henikoff et al., Proc. Natl. Acad. Sci. USA 97:716-721 (2000), incorporated herein by reference). In some in situ staining experiments, cells were heat-shocked for 2 hours at 37° C., followed by 5 hours recovery at 25° C. prior to fixation. It situ staining of proteins was carried out as described (van Steensel et al., J. Cell. Sci. 108:3003-3011 (1995), incorporated herein by reference) with C1A9 antibody against HP1 (James et al., Eur. J. Cell. Biol. 50:170-180 (1989), incorporated herein by reference), a rabbit antiserum against Cid (Henikoff et al., Proc. Natl. Acad. Sci. USA 97:716-721 (2000), incorporated herein by reference) or monoclonal antibody 9E10 against the myc-epitope tag (Santa Cruz Biotechnology, Santa Cruz, Calif.). For in situ detection of ^{m6}A in interphase cells, transfected Kc cells were grown on glass coverslips, fixed in methanol/acetic acid (3:1) for 10 minutes, washed in 70% ethanol followed by 2× SSC, denatured in 70% formamide in 2× SSC at 80° C. for 10 minutes, washed in phosphate buffered saline, and stained with antibody RI 280 (Bringmann and Luhrmann, FEBS Lett. 213:309-315 (1987), incorporated herein by reference) following the same procedure as for proteins. Mitotic spreads were prepared by incubating harvested cells 12 minutes in 1% sodium citrate at room temperature, followed by fixation in methanol/acetic acid. Fixed cells were spread on coverslips, air-dried, and stained as described above, except that denaturation was carried out for 1 minute at 60° C.

Fly lines and germline transformation: Drosophila fly lines were kept at 25° C. The GALDam1 line was established by injection of w¹¹¹⁸ embryos with pGALDam and prπ25.7wcΔ2-3 as a transposase source. One w⁺ line (GALDam1) carrying a homozygous lethal insertion on chromosome 3 was recovered. The region flanking the EP(2)0750 insertion has been sequenced (Berkeley Drosophila Genome Project, GenBank accession AC005672). It was found by PCR analysis that a blastopia element in the y²:cn bw sp strain inserted approximately 300 bp 3′ of the EP insertion position, was not present in the EP(2)0750 line. Both Me4 (which expresses Dam under a UAS-containing promoter (Wines et al., Chromosoma 104:332-340 (1996)) and GALDam1 display low but sufficient constitutive expression under uninduced conditions.

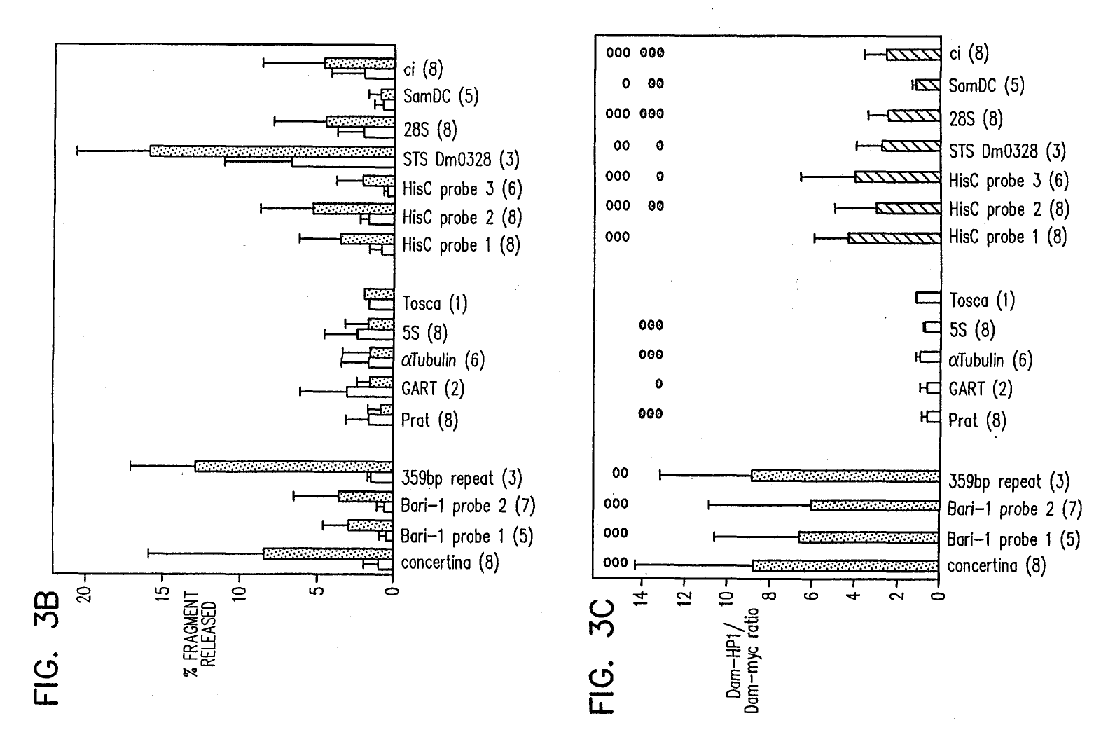

Quantitation of methylation by Southern blotting: Genomic DNA was isolated from transfected Kc cells and digested to completion with DpnI or DpnII (sew England Biolabs, Beverly, Mass.). Equal amounts of digested DNA (typically 2 to 4 μg) were separated on 1.5% agarose gels, transferred to nylon membranes, and probed with ³²P-labeled DNA probes. The hybridization intensities of DpnI-released fragments were quantitated by PhosphorImager analysis. The percentage fragment released (FIG. 3D) was calculated by normalization to an equal amount of DpnII-digested DNA from cells transfected with empty vector. The low level of fragment release was attributed, in part, to the low transfection efficiency, which was 10-30% (estimated by immunofluorescence microscopy). Probes were made by PCR-amplification from Kc genomic DNA (Table 1). A mixture of two end-labeled oligonucleotides (5′ to 3′) GTTAGCACTGGTAATTAGCTGCTCAAAACAG (SEQ ID NO: 2) and AGGAGGGGGGTCATCAAAATTTGC (SEQ ID NO: 3) was used to probe the 359 bp repeat.

Quantitation of methylation by PCR: All flies were homozygous for the EP(2)0750 insertion. Methylation was measured in DNA from single flies collected within 5 hours after eclosion. Flies were crushed in 100 μl of 10 mM Tris-HCl, pH 7.5, 10 mM EDTA, 100 mM NaCl, 0.5% SDS, 100 μg/ml proteinase K, and incubated 2-3 hours at 50° C., followed by extraction with phenol/chloroform/i-amylalcohol and extraction with chloroform. Five μl of the resulting DNA preparation was incubated 16 hours at 37° C. with or without 2 units DpnII. After heat-inactivation at 80° C., samples were diluted 1:10 or 1:100 and assayed by TaqMan quantitative PCR (Li et al., Curr. Opin. Biotechnol. 9:43-48 (1998) on an ABI7700 Sequence Detection System (PE Biosystems, Foster City, Calif.) according to the manufacturer's recommendations. Fluorogenic oligonucleotides were obtained from Synthegen (Houston, Tex.). A standard dilution series of genomic DNA from w; EP(2)0750 flies was included in every experiment to allow relative quantitation of each sample. PCR primers were chosen to flank one single GATC.

Results

In vivo targeting of Dam to a specific DNA sequence: It has been demonstrated that a DNA cytosine methyltransferase can be targeted in vitro to a specific DNA sequence by tethering it to a DNA-binding protein (Xu et al., Nat. Genet. 17:376-378 (1997)). A similar approach was tested to demonstrate whether it could be used to target E. coli DNA adenine methyl transferase to a specific DNA locus in vivo in D. melanogaster. DNA adenine methyl transferase methylates the N⁶-position of adenine in the nucleotide sequence GATC, which occurs on average every 200-300 bp in the fly genome. DNA adenine methyl transferase (DAM) was chosen because endogenous methylation of adenine does not occur in DNA of most eukaryotes. Moreover, Dam is active when expressed in yeast (Gottschling, Proc. Natl. Acad. Sci. USA 89:4062-4065 (1992); Singh et al., Genes Dev. 6:186-196 (1992); Kladde et al., Proc. Natl. Acad. Sci. USA 91:1361-1365 (1994)) and Drosophila (Wines et al., Chromosome 104:332-340 (1996)) and has no detectable effects on Drosophila development or viability (Wines et al., Chromosoma 104:332-340 (1996)), in contrast to certain cytosine methyltransferases (Lyko et al., Nat. Genet. 23:363-366 (1999)).

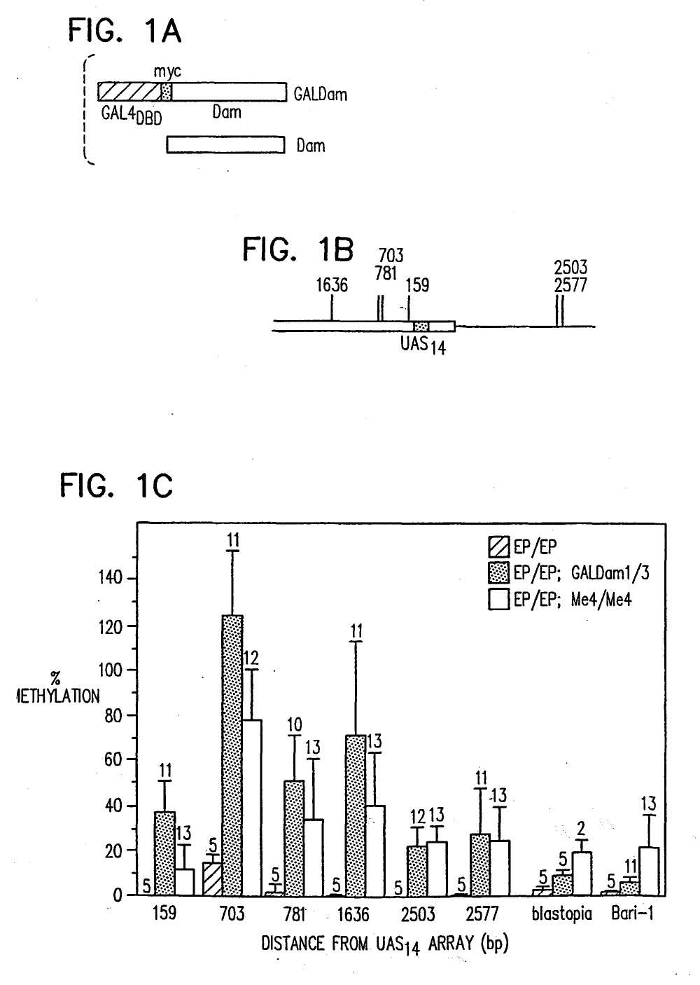

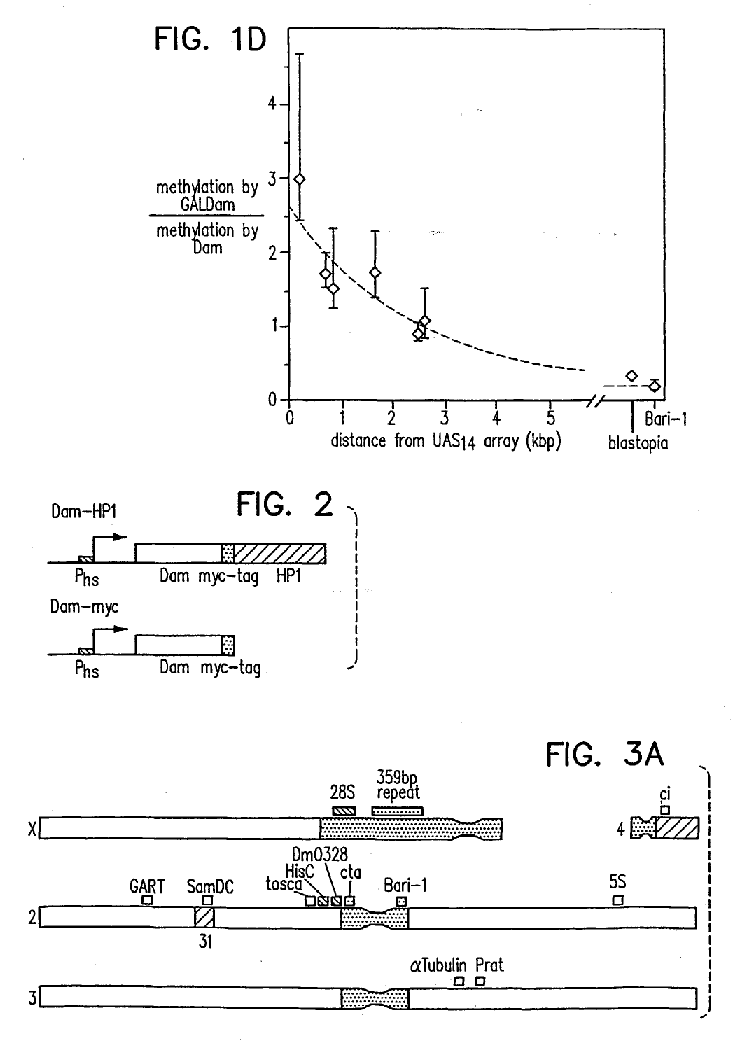

The well-characterized budding yeast protein GAL4 (Fischer et al., Nature 332:853-856 (1988)) was chosen as a DNA targeting protein. The fly line (GALDam1) was established to expresses a fusion protein (GALDam) consisting of full-length Dam and the DNA-binding domain of GAL4 (GAL4_{DBD}; FIG. 1A). A binding sequence for GAL4 was introduced by crossing GALDam1 flies to line EP(2)0750, which carries a P-element with 14 tandem binding sites for GAL4 (UAS₁₄) (Rorth, Proc. Natl. Acad. Sci. USA 93:12418-12422 (1996)) inserted into a sequenced region of chromosome 2 (FIG. 1B). As a control, EP(2)0750 was crossed to the fly line Me4, which expresses Dam alone (Wines et al., Chromosoma 104:332-340 (1996)). The progenies of these crosses were used to test whether GAL4_{DBD} was able to target Dam to GATCs in the vicinity of the UAS 14 array.

The methylation frequencies of individual GATC sequences was determined using an assay based on quantitative PCR (Li et al., Curr. Opin. Biotechnol. 9:43-48 (1998)). This sensitive assay allowed for the measurement of methylation levels in single flies. Several GATC sequences at various distances from the UAS₁₄ array (FIG. 1B and FIG. 1C) were tested. Because local differences in chromatin accessibility affect the methylation frequency of individual GATC sequences (Gottschling, Proc. Natl. Acad. Sci. USA 89:4062-4065 (1992); Singh et al., Genes Dev. 6:186-196 (1992); Kladde et al., roc. Natl. Acad. Sci. USA 91:1361-1365 (1994); Wines et al., Chromosoma 104:332-340 (1996)), the ratio of methylation by GALDam and methylation by Dam was calculated for each GATC. These ratios were plotted as a function of the distance from the UAS₁₄ array (FIG. 1D).

To determine the level of non-targeted ‘background’ methylation by freely diffusing GALDam protein, the methylation levels of two remote loci was first measured. For this GATC sequences were chosen in the pericentric Bari-1 element (Caizzi et al., Genetics 133:335-345 (1993)), which is located more than 10 Mb away, and in the blastopia element, which was present in 10-20 copies scattered throughout the genome (Frommer et al., Chromosoma 103:82-89 (1994)). In these remote loci the methylation ratio (GalDam1/Me4) were about 0.2-0.3 (FIG. 1C and FIG. 1D). This value presumably reflects the relative nuclear concentrations and methyltransferase activities of the two proteins. Strikingly, GATC sequences in the vicinity of the UAS₁₄ array showed GalDam1/Me4 methylation ratios in the range of about 1 to 3. This five- to ten-fold increase clearly indicated that methylation by GALDam was targeted to the vicinity of the UAS₁₄ array. This targeted methylation was significant for each of six tested GATC sequences within approximately 2.5 kb from the UAS₁₄ array, when compared to the Bari-1 GATC (P<0.05, t-test for ratios (Goldstein, Biostatistics. An introductory text, MacMillan Co., New York (1964)). Fitting of an exponential curve to the data in FIG. 1D indicated a gradient of targeted methylation (r=0.89; P<0.02), with most targeted methylation occurring within roughly 5 kb from the UAS₁₄ array. These data demonstrated that methylation by Dam could be targeted in vivo to the vicinity of a specific DNA sequence.

Targeting of Dam by Heterochromatin Protein 1 (HP1): Whether targeted methylation could be used to identify binding sites of an endogenous chromatin protein was investigated. Antibody staining of Drosophila polytene chromosomes has previously indicated that HP1 was predominantly associated with heterochromatin (James et al., Eur. J. Cell. Biol. 50:170-180 (1989)). HP1 antibodies also decorate most of chromosome 4 and a number of euchromatic loci. It has been thought that HP1 is recruited to specific genomic loci by other chromatin proteins (Platero et al., EMBO J. 14:3977-3986 (1995)). To identify HP1 target loci, a myc-epitope tagged Dam-HP1 fusion protein construct driven by a heat-shock promoter (FIG. 2) was transfected into Drosophila Kc cells, and the resulting methylation patterns were mapped. As a control. myc-tagged Dam (Dam-myc) was expressed.

In order to demonstrate that fusion to Dam did not impair correct targeting of HP1, advantage was taken of the observation that HP1 in Kc cells was predominantly located in a large discrete compartment in the nucleus. This compartment represented clustered pericentric heterochromatin of all chromosomes, since all centromeres were generally located within this compartment. In agreement with this, AT-rich heterochromatic satellite repeats, which were visible as DAPI-bright regions, were closely associated with the HP1 compartment. HP1 was also seen in a few small brightly stained dots scattered throughout the nucleoplasm.

After heat shock induction, the Dam-HP1 fusion protein (detected with an antibody against the myc epitope) showed a subnuclear distribution pattern that strongly resembled that of endogenous HP1. Both the large compartment, closely associated with the DAPI-bright regions, and a few small dots scattered throughout the nucleus were observed. This indicated that the fusion protein was correctly targeted to natural HP1 binding sites. In contrast, after heat-shock induction the Dam-myc protein showed a very weak staining throughout the cell, with no indication of subnuclear targeting. In the absence of heat shock induction the Dam-HP1 or Dam-myc proteins were not detectable by immunofluorescence, indicating very low expression levels under those conditions.

Whether expression of Darn-HP1 leads to preferential methylation of heterochromatic DNA was also tested. Sites of methylation were visualized in situ using a rabbit antiserum (Bringmann et al., FEBS Lett. 213:309-315 (1987)) against methyl-N⁶-adenine (^{m6}A). After heat-shock induction, cells transfected with either Dam or Dam-HP1 showed strong ^{m6}A staining throughout the nucleus. Cells transfected with an empty vector showed no nuclear staining. Strikingly, in the absence of heat shock induction, cells transfected with Dam-HP1generally showed staining of the large, discrete nuclear compartment associated with DAPI-bright regions. Co-staining of ^{m6}A with an antibody against endogenous HP1 confirmed that methylation was mostly restricted to the heterochromatic compartment. An imprecise correspondence of the two staining patterns was expected because of a lack of GATCs in the simple satellites in Drosophila heterochromatin. In metaphase chromosomes of cells transfected with Dam-HP1, ^{m6}A was highly enriched in pericentric regions and in the heterochromatic proximal half of the X-chromosomes, further confirming preferential methylation of heterochromatin.

It can be concluded that under non-heat shock conditions, the Dam-HP1 fusion protein was present at levels that were too low to be detected by immunocytochemistry, yet were sufficient to result in specific methylation of heterochromatic DNA. High protein expression (after heat-shock) led to high levels of non-targeted methylation throughout the nucleus. No ^{m6}A-labeling was observed in the absence of heat shock induction in cells transfected with Dam-myc, presumably because methylation levels were below the detection limit of the in situ staining. Taken together, these data indicate that Dam-HP1 was correctly targeted to native binding sites of HP1 (i.e., heterochromatin), and that targeting results in preferential methylation of these sites provided that the fusion protein is expressed at low levels.

Identification of targets for HP1 at the sequence level: Targeted methylation was tested to determine if it could be exploited to identify genomic loci that are associated with HP1. Quantitative Southern blot assay (Boivin et al., Genetics 150:1539-1549 (1998), incorporated herein by reference) was used to determine the methylation level of pairs of GATCs in individual loci. Initially, methylation levels were quantitated in four known heterochromatic and five known euchromatic regions (FIG. 3A and FIG. 3B). After transfection with Dam-HP1, euchromatic loci showed similar levels of methylation as after Dam-myc transfection (76±23% of Dam-myc values). This indicated that the methyltransferase activity of Dam-HP1 was comparable to that of Dam-myc.

In contrast, heterochromatic loci displayed much higher methylation levels in cells transfected with Dam-HP1 than in cells transfected with Dam-myc. Direct comparison of methylation levels of each locus by Dam-HP1 and Dam-myc (by calculating the ratio Dam-HP1 methylation/Dam-myc methylation; FIG. 3C), showed a clear and consistent distinction between eu- and heterochromatic loci. Dam-HP1/Dam-myc methylation ratios were approximately 10-fold higher in heterochromatic regions compared to euchromatin regions (p<0.0001, Mann-Whitney U-test). Thus, methylation by Dam-HP1 was determined to be a good criterion for the identification of genomic loci that are associated with HP1 in vivo.

A number of additional loci were characterized that might be expected to interact with HP1, but for which no direct evidence was available. The histone gene cluster repeat (HisC), located in euchromatin close to the pericentric heterochromatin of chromosome 2, has been speculated to have heterochromatic features (Fitch et al., Chromosoma 99:118-124 (1990)). The rDNA repeat has been mapped to the heterochromatic part of the X chromosome (Hilliker et al., Cell 21:607-619 (1980)), yet during interphase it is packaged inside the transcriptionally active nucleolus (Scheer et al., Opin. Cell. Biol. 11:385-390 (1999)). Somewhat surprisingly, it was found that both loci (three different regions in HisC, and the 28S gene in the rDNA repeat) displayed Dam-HP1/Dam methylation ratios that were intermediate between euchromatin and heterochromatin (FIG. 3C). Since both loci are tandem repeats, it is possible that only a fraction of the repeats was associated with HP1. Alternatively, the association of HP1 with these loci may be cell cycle regulated. A similar level of Dam-HP1 targeted methylation was observed for sequence tag STS Dm0328, which is located in the banded region proximal to HisC. Possibly, HP1 ‘spreads’ from pericentric heterochromatin into the flanking euchromatin to include HisC.

Finally, the cubitus interruptus (ci) and S-adenosyl decarboxylase (SamDC) genes were tested. These genes are located in the banded part of chromosome 4 and in region 31 on chromosome 2, respectively. Both regions are decorated by antibodies against HP1 (James et al., Eur. J. Cell. Biol. 50:170-180 (1989)). ci showed levels of HP1-targeted methylation that were lower than in heterochromatic loci, but significantly higher than in euchromatic loci (FIG. 3B and FIG. 3C), indicating that HP1 is associated with this gene. In contrast, SamDC showed low levels of targeted methylation, suggesting that this gene was not abundantly associated with HP1. A detailed map of HP1 associations can be obtained in the future by systematic analysis of a large number of sequences throughout the genome.

Discussion

The data provided herein demonstrate that DamID can be used to identify sequences that interact in vivo with specific proteins. Targeting of Dam leads to up to an approximately 10-fold enrichment of methylation in the vicinity of binding sites of the Dam fusion partner, which is sufficient for positive identification of most target sequences, and for detecting quantitative differences -in protein-target interactions. The ‘background’ methylation throughout the genome by Darn fusion proteins was attributed to the intrinsic DNA binding activity of Dam, which would compete with the sequence- or locus-specific interactions of its fusion partner. However, it is also possible that chromatin proteins are rather promiscuous in their interactions in vivo.

DamID has a number of important advantages over other current methods for identification of target sequences. First, DamID detects protein-DNA interactions as they occur in living cells. Other approaches, such as the use of cross-linking reagents (reviewed in Simpson, Curr. Opin. Genet. Dev. 9:225-229 (1999)) or targeted nucleases (Lee et al., Proc. Natl. Acad. Sci. USA 95:969-974 (1998)) may induce alterations in the chromatin structure under investigation. Adenine methylation has only minor effects on DNA topology (Barras et al., Trends Genet. 5:139-143 (1989)), which was underscored by the observation that fly development was not affected even when approximately 50% of all GATCs in the genome were methylated (Wines et al., Chromosoma 104:332-340 (1996); Boivin et al., Genetics 150:1539-1549 (1998)).

Moreover, it was found herein that DamID works best when the Dam fusion protein was expressed at very low levels, making it unlikely that the fusion protein itself interfered with the functions of the endogenous protein or its targets. Second, DamID can be used both in cell cultures and in whole organisms. Since endogenous methylation of adenine is not detectable in DNA from most eukaryotes, this technique should be widely applicable. Preliminary results indicate that Dam is active, yet has no obvious toxic effects, when expressed in HeLa cells. Third, using the PCR-based quantitation assay, detection of protein-DNA interactions was extremely sensitive. The data provided herein demonstrated that DamID can be applied in single flies, and it can be anticipated that smaller tissue samples or perhaps only a few cells could be analyzed with this assay.

TABLE 1
Exact positions of probed GATC pairs in FIG. 3.

| Locus^{a} | GenBank accession | positions |
|---|---|---|
| concertina | M94285^{b} | 827, 1124 |
| Bari-1 probe 1(R) | X67681 | 1607, 195 |
| Bari-1 probe 2 (R) | X67681 | 195, 548 |
| 359bp repeat (R) | V00225 | 96, 53 |
| Prat | AF017096 | 5742, 6567 |
| GART | X06286 | 3348, 3663 |
| aTubulin | M14643 | 1879, 2345 |
| 5S (R) | X87880 | 228, 228 |
| tosca | X89022 | 3134, 3722 |
| HisC probe 1 (R) | X14215 | 688, 1127 |
| HisC probe 2 (R) | X14215 | 3835, 668 |
| HisC probe 3 (R) | X14215 | 2490, 3835 |
| STS Dm0328 | BDGP^{c} | 135, 393 |
| 28S (R) | M21017 | 5018, 5492 |
| SamDC | Y11216 | 410, 961 |
| ci | U66884 | 12262, 12865 |

^{a}(R) indicates that the probed region is predominantly present as a tandem repeat array
^{b}cDNA sequence; the corresponding genomic sequence (GenBank accession AF211849) contains two approximately 50 bp introns
^{c}Berkeley Drosophila Genome Project database

### EXAMPLE 2

The present example provides large-scale mapping of in vivo binding sites of chromatin proteins, using a combination of targeted DNA modification and microarray detection. Three distinct chromatin proteins in Drosophila Kc cells were mapped and each were found to associate with specific sets of genes. HP1 was found, as above, binds predominantly to pericentric genes and transposable elements, GAGA factor associates with euchromatic genes that are enriched in (GA)ₙ motifs. Surprisingly, a Drosophila homolog of yeast Sir2 was found to associate with several active genes and was excluded from heterochromatin.

The materials and methods used for microarray detection of modified DNA regions peripheral to the binding, loci of the DNA binding protein were as follows.

Plasmids: Vectors for expression of myc-tagged Dam and Dam-HP1 were described above. A cDNA encoding full-length DmSir2-1 (GenBank accession AF068758) was obtained by PCR amplification from a Drosophila ovary cDNA library and cloned into pCMycDam as above, resulting in plasmid pSir2α-MDam. Sequencing of the cloned PCR product revealed that six nucleotides encoding Phe₂₈₉ and Gln₂₉₀ were missing. The same polymorphisms were also present in a genomic sequence (Genbank AE003639). Dam was fused to the C-terminus of DmSir2-1 because it had previously been found that addition of Green Fluorescent Protein to this end of yeast Sir2 did not interfere with correct subnuclear targeting of Sir2 (Cuperus et al., EMBO J. 19:2641-2651 (2000)).

Full length GAF (the 519 amino acid isoform (Benyajati et al., Nucleic Acids Res. 25:3345-3353 (1997), incorporated herein by reference) was amplified from plasmid pAR-GAGA (Soeller et al., Mol. Cell. Biol. 13:7961-7970 (1993)) and cloned either into pCMycDam (resulting in pGAF-MDam) or pNDamMyc (resulting in pDamM-GAF).

Transfections and DNA purification: Drosophila Kc cells were grown and transfected as described by Henikoff et al. (Proc. Natl. Acad. Sci. USA 97:716-721 (2000), incorporated herein by reference). Expression of the Dam proteins was driven by the low constitutive activity of the uninduced hsp70 promoter, ensuring very low expression levels. After 24 hours, genomic DNA was isolated as described by de Lange et al. (Mol. Cell. Biol. 10:518-827 (1990), incorporated herein by reference), except that an RNase incubation was included before the second proteinase K treatment. About 0.5 to 1.5 mg of the isolated DNA (from approximately 10⁹ cells) was digested for 16 hours with 500 to 1,000 units of DpnI (New England Biolabs) and size-fractionated by ultracentrifugation in a Beckman SW-40T1 swing-out rotor for 16 hours at 79,000×g on 11 ml sucrose gradients (5 to 30%) containing 10 mM Tris-HCl (pH7.4), 150 mM NaCl, 10 mM EDTA. Gradient fractions containing DNA fragments smaller than about 2.5 kb (as judged by agarose gel electrophoresis) were pooled and concentrated by isopropanol precipitation.

Typically, this procedure yielded 20 to 50 μg methylated DNA. Genomic DNA from control cells transfected without plasmid was processed in parallel and generally gave a 5 to 10 fold lower yield of <2.5 kb fragments, indicating that the methylated DNA was about 80 to 90% pure. An estimated 20 to 50% of the methylated DNA consisted of plasmid DNA that was taken up by the cells during transfection. This plasmid DNA does not interfere with the subsequent labeling and hybridization procedure.

Microarrays and hybridizations: Microarray construction and hybridization protocols were modified and optimized from those described elsewhere (DeRisi et al., Science 278:680-686 (1997), incorporated herein by reference). Briefly, Drosophila microarrays were constructed employing a set of 192 cDNAs randomly selected from the LD and CK EST libraries (Berkeley Drosophila Genome Project) (Rubin et al., Science 287:2222-2224 (2000); Kopczynski et al., Proc. Natl. Acad. Sci. USA 95:9973-9978 (1998)) (Research Genetics, Huntsville, Ala.), together with about 140 cDNA and about 20 genomic DNA fragments provided by members of the Northwest Fly Consortium. Each clone insert was individually PCR-amplified and each product was verified as unique via gel electrophoresis. PCR products were purified using Arraylt 96-well PCR purification kits (TeleChem International, Sunnyvale, Calif.) and mechanically “spotted” in 3× SSC (450 mM NaCl and 45 mM sodium citrate, pH 7.0) onto poly-lysine coated microscope slides using an OmniG rid high-precision robotic gridder (GeneMachines, San Carlo, Calif.).

One μg of purified methylated DNA was labeled with Cy3- or Cy5-dCTP (Amersham Pharmacia, Piscataway, N.J.) by random priming (Pollack et al., Nature Genet. 23:41-46 (1999)). Labeled experimental and reference DNA samples were mixed and hybridized to a microarray in 3× SSC in the presence of 20 μg poly [dA.dT], 100 μg yeast tRNA, and 25 μg unlabeled DpnI-digested plasmid encoding the fusion protein that was used for transfection. Hybridization was performed at 63° C. for 16 hours followed by sequential washings in 1× SSC, 0.03% SDS, 1× SSC, 0.2× SSC, and 0.05× SSC. Washed arrays were spun dry in a centrifuge and immediately scanned using a GenePix 4000 fluorescent scanner (Axon Instruments, Inc., Foster City, Calif.).

Data analysis: Image processing was performed using GenePix 3.0 image analysis software. Statistical analysis was performed using StatView software (Abacus Concepts, Berkeley, Calif.). Cy3:Cy5 ratios were normalized using Drosophila total genomic DNA (spotted 16 times on each microarray slide) as an internal standard. Thus, a Cy3:Cy5 value of 1 represents the average level of binding of the chromatin protein along the entire genome.

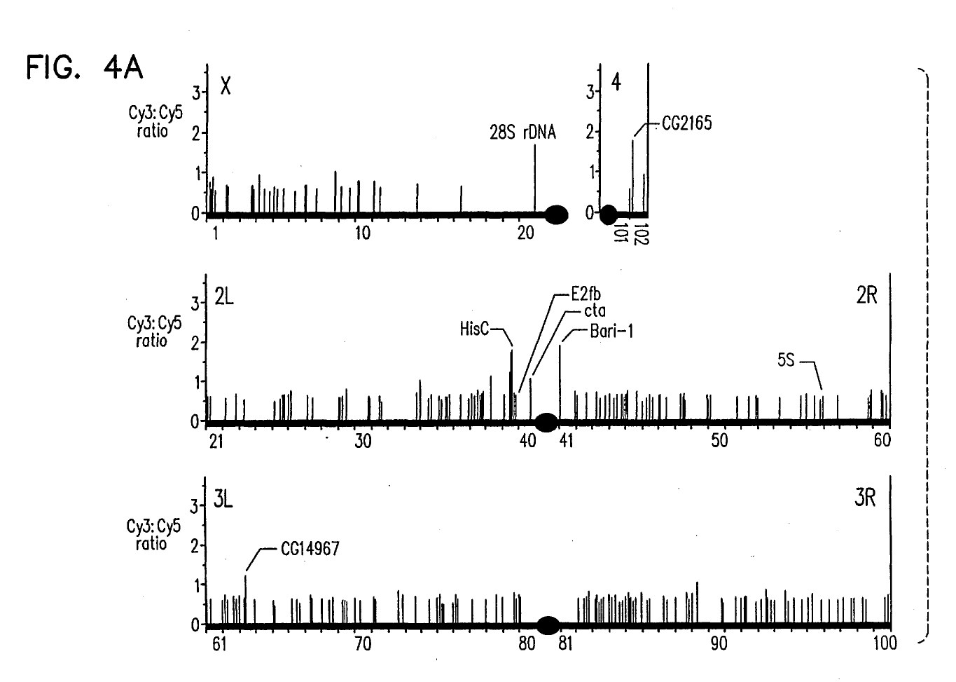

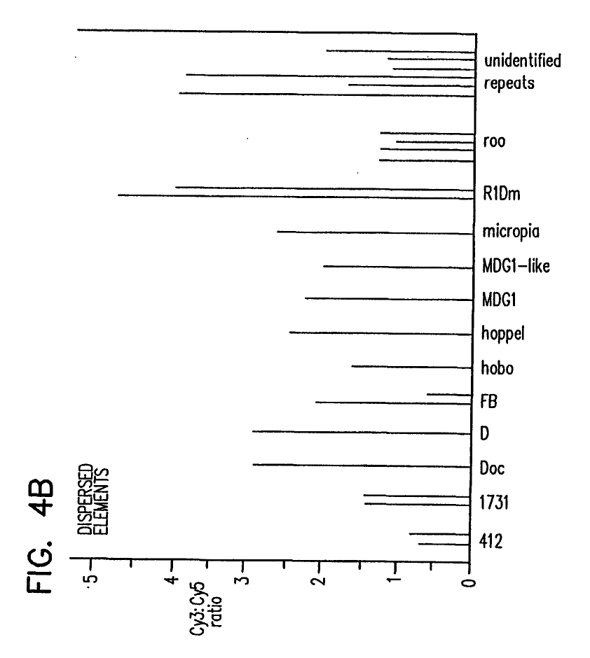

Five ESTs initially thought to represent unique genes along the euchromatic arms (based on the available 5′ sequence) were identified as HP1 targets. Sequencing of both 5′ and 3′ ends indicated that these clones are hybrids of a unique gene and a repetitive sequence. It was presume that this was a cloning artifact that occurred during the construction of the CK library (Kopczynski et al., Proc. Natl. Acad. Sci. USA 95:9973-9978 (1998)). These clones are represented in the Dispersed Elements section of FIGS. 4 through 6. The fact that these chimeric clones were identified as HP1 targets underscores the sensitivity of the assay of the present invention.

Analysis of the density of (GA)ₙ elements was carried out as follows. Fifteen loci showing low GAF-Dam binding (Cy3:Cy5 ratios 0.97±0.09) and 15 loci showing high GAF-Dam binding (Cy3:Cy5 ratios 2.77±0.65) were selected based on availability of the complete probe sequence. Corresponding genomic sequences were obtained from the BDGP/Celera genomic database and covered the region encoding the cDNA fragment present on the microarray. Any introns smaller than 5 kb, as well as 3 kb of sequence upstream and downstream of the probed region were included in the analysis, because methylation by tethered Dam extends in cis over a few kbs. On average, about 7.5 kb was analyzed per probed region. GAGAG (SEQ ID NO: 4) and GAGAGAG (SEQ ID NO: 5) sequences were counted in both orientations; partially overlapping elements were counted separately (e.g., the sequence TGAGAGAGC (SEQ ID NO: 6) contains two GAGAG (SEQ ID NO: 4) and one GAGAGAG (SEQ ID NO: 5) element).

Immunocytochemistry: For in situ staining, Kc cells were transfected with plasmids encoding Dam-fusion protein under a heat-shock inducible promoter and grown on microscope coverslips. After 18 hours, cells were heat-shocked at 37° C. for 2 hours, followed by 5 hours recovery at 25° C. Immunocytochemistry was performed as described (Henikoff al., Proc. Natl. Acad. Sci. USA 97:716-721 (2000), incorporated herein by reference), using a rabbit polyclonal antibody against the hinge domain of HP1. Dam fusion proteins were detected using mouse monoclonal antibody 9E10 (Santa Cruz Biotechnology, Santa Cruz, Calif.) against the myc epitope, which is present as a linker peptide between Dam and its fusion partners.

### RESULTS

Drosophila Kc cells were transfected either with a Dam fusion protein or with Dam only. The latter served as a reference to normalize for local differences in chromatin accessibility to methylation by Dam. Twenty-four hours after transfection, genomic DNA was isolated and methylated regions were purified. Purified methylated DNA samples from experimental and control cells were labeled with the fluorochromes Cy3 and Cy5, respectively, mixed, and co-hybridized to microarrays of approximately 300 Drosophila cDNAs, most of which were randomly chosen from two different embryonic cDNA libraries. Target sequences of the chromatin proteins of interest were identified based on the Cy3:Cy5 fluorescence ratio, which indicated the relative targeted methylation level of each probed sequence.

Mapping of target loci of HP1: Heterochromatin Protein 1 (HP1) was selected to test the mapping technique of the present invention because of its unique chromosomal distribution. Immunocytochemistry of polytene chromosomes has indicated that HP1 is predominantly associated with pericentric heterochromatin, and additionally with chromosome ends and several bands scattered throughout the euchromatic arms (James et al., Eur. J. Cell Biol. 50:170-180 (1989); Fanti et al., Mol. Cell 2:527-538 (1998)). HP1 is thought to be recruited to its target loci by other chromatin proteins (Platero et al., EMBO J. 14:3977-3986 (1995); Lehming et al., Proc. Natl. Acad. Sci. USA 95:7322-7326 (1998); Ryan et al., Mol. Cell. Biol. 19:4366-4378 (1999)). A small number of pericentric target loci of HP1 have been identified (van Steensel and Henikoff, Nature Biotechnol. 18:424-428 (2000)), but the nature of the HP1 binding sites on the euchromatic arms is unknown.

A scatter diagram of the hybridization signals measured for Cy3 (Dam-HP1) vs Cy5 (Dam) showed that the majority of cDNAs display an almost identical Cy3:Cy5 ratio (i.e., were located on a single diagonal in the scatter diagram), indicating no detectable association of the corresponding genes with HP1. However, a distinct set of cDNAs demonstrated a clear offset from this diagonal towards higher Cy3:Cy5 ratios. These cDNAs must represent target loci of HP1. The absence of data points with lower Cy3:Cy5 ratios demonstrated that tethering of Dam to HP1 caused an increase in methylation of HP1 target loci, but not a decrease in methylation levels of non-target loci.

The probed loci are represented in FIG. 4A on the standard polytene chromosome map, showing their relative HP1 binding (i.e., Cy3:Cy5 ratios). Most loci display a constant Cy3:Cy5 ratio (approximately 0.5-0.6), which was interpreted as non-targeted ‘background’ methylation. However, several loci demonstrated a considerably higher ratio, implying HP1 binding. Although the cutoff between ‘target’ and ‘non-target’ Cy3:Cy5 ratios was arbitrary, it is important to note that the differences in Cy3:Cy5 ratios between probed loci were highly reproducible. Pair-wise comparisons of three independent experiments showed correlation coefficients between 0.95 and 0.99. Hence, loci that demonstrated only a mild increase in Cy3:Cy5 ratio over background levels (e.g., gene CG14967, FIG. 4A) were likely to be associated with HP1 in vivo, although the local HP1 concentration may be lower than at other target loci with higher Cy3:Cy5 ratios. Moreover, differences in Cy3:Cy5 ratios between genes in the present assay may somewhat underestimate differences in protein binding. By Southern blot analysis it was found that the Ban-1 locus displays about 8-fold higher HP1-targeted methylation than the 5S rDNA locus (Example 1), yet the present microarray analysis indicated only about a 4-fold difference. Such a microarray-specific compression effect has been observed previously (Pollack et al., Nature Genet. 23:41-46 (1999)).

Among the target loci of HP1 detected were genes located near pericentric heterochromatin, or on the largely heterochromatic chromosome 4. Both the histone gene cluster (HisC) and the cta gene, located near the centromere on the left arm of chromosome 2, were found to be associated with HP1, in agreement with previous observations provided above in Example 1. In contrast, the Ef2b gene, which lies between these two loci, showed no detectable HP1 binding, suggesting a discontinuous distribution of HP1 in this region. This was consistent with a banded pattern of HP1 staining in this region in polytene chromosomes. Interestingly, at least one gene located on one of the euchromatic chromosome arms (gene CG14967) demonstrated an association with HP1 (FIG. 4A), although this association may be relatively weak.

In addition, HP1 was found to bind to a wide variety of transposable elements. Of 12 different transposons present on the microarray, 11 showed moderate to strong association with HP1 (FIG. 4A). HP1 binding was consistent with the enrichment of most transposable elements in pericentric heterochromatin (Charlesworth et al., Genet. Res. 64:183-197 (1994); Pimpinelli et al., Proc. Natl. Acad. Sci. USA 92:3804-3808 (1995); Carmena et al., Chromosoma 103:676-684 (1995)). The 412 element demonstrated no detectable HP1 association. The lack of HP1 binding to this element was unexpected, since it has been determined to be closely related to the MDG1 element, which demonstrated a strong HP1 association. In some Drosophila strains the 412, element demonstrated preferential insertion in euchromatin rather than in heterochromatin (Charlesworth et al., Genet. Res. 64:183-197 (1994)), and it might be that most 412 copies in the genome of Kc cells are distant from heterochromatic domains.

Target loci of GAF: Similar assays were performed with GAF as the targeting protein to investigate the general applicability of the mapping technique of the present invention. GAF is different from HP1 in many ways. For example, GAF binds directly to GA-rich DNA sequences, and has been implicated in the regulation of several Drosophila genes (Wilkins et al., Nuc. Acids Res. 25:3963-3968 (1997); Granok et al., Curr. Biol. 5, 238-241 (1995)). Immunocytochemistry of polytene chromosomes has indicated that GAF has hundreds of euchromatic binding sites during interphase (Tsukiyama et al., Nature 367:525-532 (1994); Benyajati et al., Nuc. Acids Res. 25:3345-3353 (1997)), although association of GAF with GA-rich heterochromatic satellite repeats has been observed during mitosis (Platero et al., J. Cell Biol. 140:1297-1306 (1998)). Most of the euchromatic binding sites have not been identified at the sequence level.

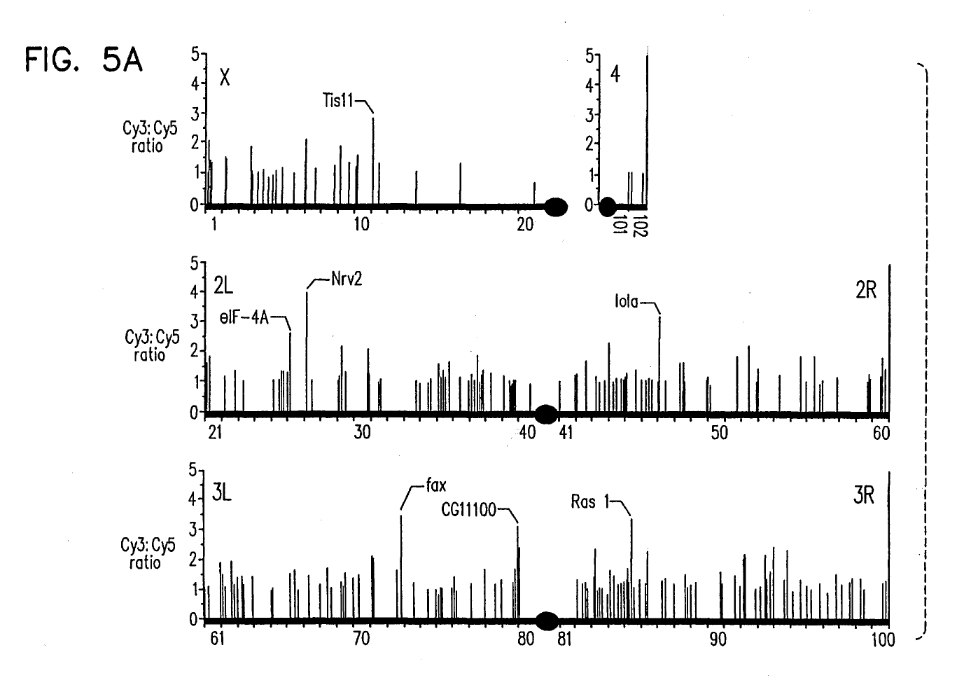

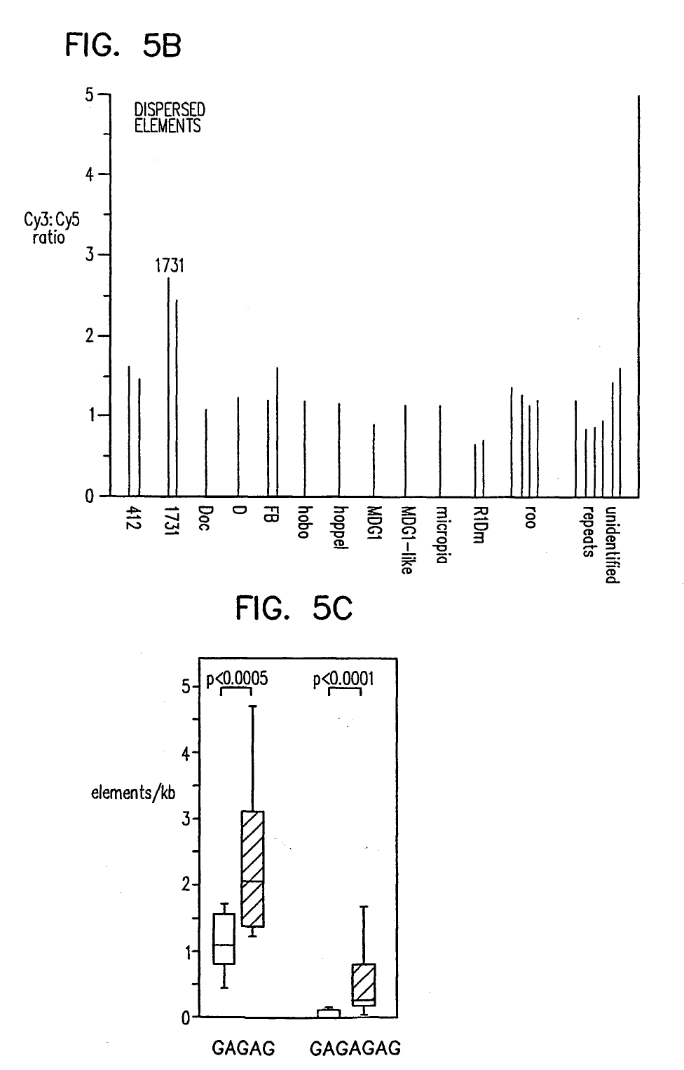

GAF target loci were mapped using a fusion protein (GAF-Dam) comprising Dam linked to the C-terminus of full-length GAF. A chromosomal map of GAF-Dam binding (FIG. 5A) indicated that GAF interacts with many genes, although to varying degrees. The variation between genes was not entirely due to random noise, because correlation analysis of independent experiments demonstrated that the results were highly, reproducible (r=0.90). Moreover, identical assays were carried out with Dam fused to the N-terminus of GAP. Again, results were reproducible, with r in pairwise comparisons of three experiments ranging from 0.81-0.93. Importantly, the C- and N-terminal fusion proteins gave similar results, r=0.80, in two independent comparisons), although Cy3:Cy5 ratios with Dam-GAP cover a smaller dynamic range than with GAF-Dam. These results strongly suggest that Dam, when fused to either end of GAF, does not interfere with correct targeting of GAF.

Genes that appear to strongly bind GAF have no common function or expression pattern. Because in vitro binding assays and in vivo cross-linking studies have shown that GAF binds GA-rich regulatory elements (Biggin et al., Cell 53:699-711(1988); Soeller et al., Mol. Cell. Biol. 13:7961-7970 (1993); Strutt et al., EMBO J. 16:3621-3632 (1997); O'Brien et al., Genes Dev. 9:1098-1110(1995)), the GAF target loci identified by the mapping were investigated to determine whether they were enriched in such elements. Indeed, loci that display moderate to strong GAF binding have significantly higher average densities of GAGAG (SEQ ID NO: 4) and GAGAGAG (SEQ ID NO: 5) sequences than loci with low GAF binding (FIG. 5B) providing strong evidence that bona fide target loci of GAF were identified.

Target loci of DmSir2-1: Finally, target loci of a Drosophila homolog of budding yeast Sir2 were mapped by the methods of the present invention. In S. cerevisiae, Sir2 plays a role in silencing of genes in the silent mating-type loci, telomeric regions, and the rDNA locus (Guarente, Genes Dev. 14:1021-1026 (2000); Gartenberg, Curr. Opin. Microbiol. 3:132-137 (2000)). In Drosophila, five Sir2-like proteins have been predicted by sequence analysis (Frye, Biochem. Biophys. Res. Commun. 273:793-798 (2000)). Of these five, the Sir2-like protein that was found to be most closely related to S. cervisiae Sir2 was chosen. The selected protein has been referred to herein as DmSir2-1. The homology to yeast Sir2 suggested that DmSir2-1 might be associated with heterochromatin in Drosophila, but no experimental studies of DmSir2-1 have been reported.

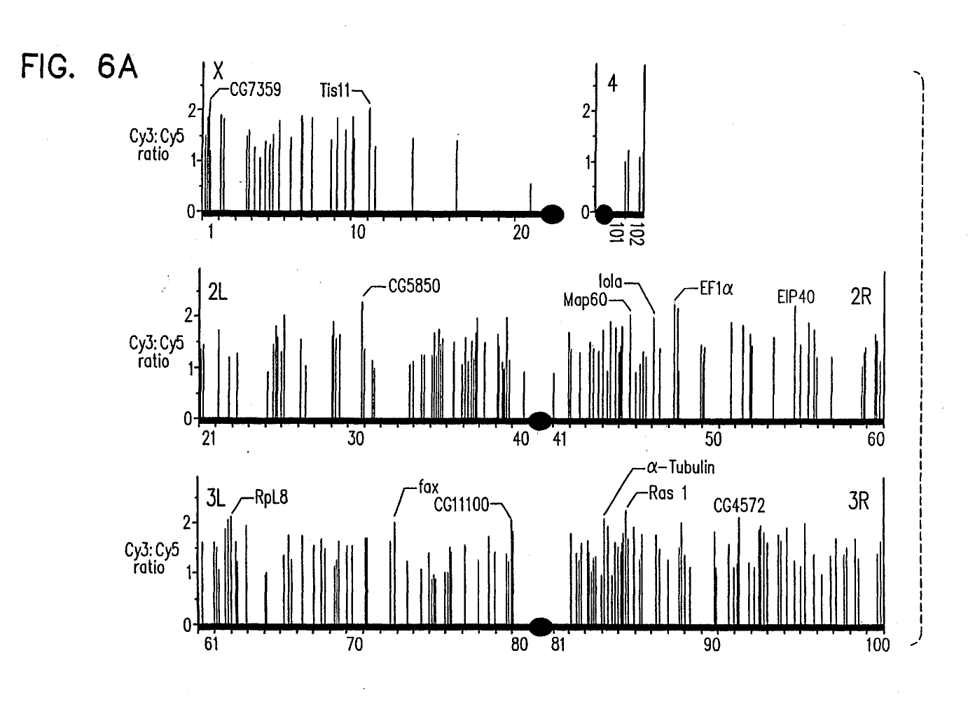

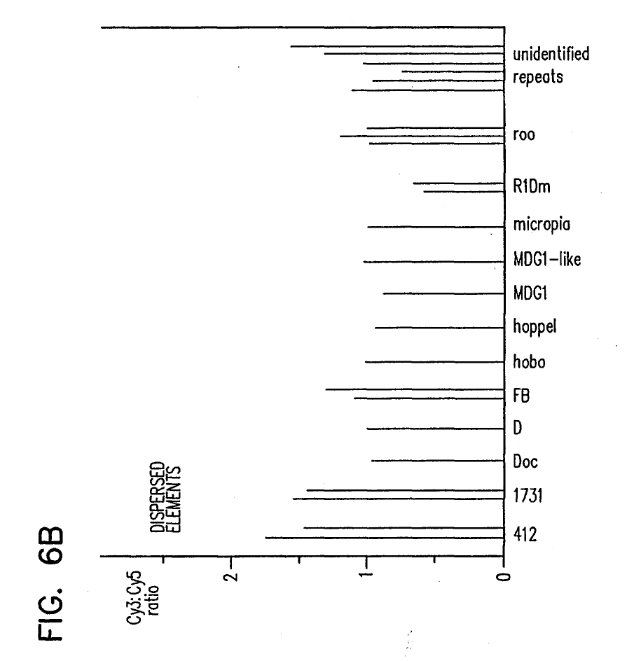

Mapping results obtained with a DmSir2-1-Dam fusion protein are shown in FIG. 6. DmSir2-1 demonstrated association with numerous genes in a reproducible fashion (r=0.81 between two independent experiments). Among the strongest DmSir2-1 binding loci were several euchromatic, constitutively expressed genes such as genes encoding translation factors, putative ribosomal proteins, α-tubulin, hsc4 and EIP40. This suggests that DmSir2-1 binds to active genes, unlike yeast Sir2.

The binding of DmSir2-1 to active genes was not simply due to the ‘open’ chromatin conformation of these genes, because the binding data were corrected for local differences in accessibility. To rule out that the protocol of the present invention somehow leads to undercorrection, local chromatin accessibility was mapped in a separate set of assays by using purified methylated DNA from cells expressing unfused Dam as the probe, and total genomic DNA as the reference probe. These assays identified several highly accessible genes that nevertheless displayed low levels of DmSir2-1 binding. These data indicate that DmSir2-1 did not generally bind to ‘open’ chromatin regions, but specifically interacted with a set of active genes.

The results also indicated that DmSir2-1 did not interact with the 28S genes in the rDNA gene cluster (FIG. 6). Again, this was in contrast to yeast Sir2, which bound to the rDNA repeat (Gotta et al., EMBO J. 16:3243-3255 (1997)). This lack of binding was not due to a general inaccessibility of the nucleolar compartment, because HP1 shows clear association with the 28S genes (FIG. 4A and in Example 1).

Comparison of the distributions of HP1, GAF, and DmSir2-1: The establishment of genome-wide maps of binding sites of several chromatin proteins in the same cell type provided the unique opportunity to compare the global binding patterns of these proteins. Such comparisons can reveal possible interactions between proteins, or demonstrate exclusion of one protein from the targets of another protein.

Relationships between the distributions of HP1, GAF, and DmSir2-1 were analyzed by creating pairwise scatter diagrams. Strikingly, comparison of the distributions of HP1 and DmSir2-1 revealed that loci with high levels of HP1 binding contained low levels of DmSir2-1. The exclusion of DmSir2-1 from HP1 targets was highly significant (p<0.0001), indicating that DmSir2-1 was not part of heterochromatin. These data, taken together with the observed association of DmSir2-1 with active genes, suggested that DmSir2-1 does not play a major role in gene silencing, in contrast to Sir2 in S. cerevisiae. Functional predictions based only on sequence homology can be misleading (Bork and Bairoch, Trends Genet. 12:425-427 (1996); Bork and Koonin, Nature Genet. 18:313-318 (1998)), and it is possible that one or more of the other four Sir2 homologs in Drosophila (Frye, Biochem. Biophys. Res. Commun. 273:793-798 (2000)) are associated with heterochromatin.

Visual comparison of GAF and HP1 distributions suggested a similar exclusion of GAF from HP1 binding sites. Such exclusion was indeed significant for GAF-Dam (p<0.003), but not for Dam-GAF (p=0.25). The somewhat higher noise level in the Dam-GAF data obtained in the assays may preclude detection of exclusion from HP1 binding sites. Finally, comparison of GAF and DmSir2-1 revealed a subset of genes that were associated with both proteins. Biochemical analysis may reveal whether the two proteins can be part of one protein complex, or whether they bound separately to different regions in the same genes.

To confirm the relative distributions of the three proteins their immunocytochemistry staining patterns were examined. As provided above in Example 1, HP1 in Kc cells was associated with a large chromocenter. In contrast, the DmSir2-1-Dam fusion protein appeared to be associated with the euchromatic compartment, and was essentially excluded from HP1-containing regions. Likewise, the GAF-Dam fusion protein was located in the euchromatin compartment and mostly absent from the chromocenter. These cytological results were in agreement with the molecular mapping data, and confirmed that GAF and DmSir2-1 were preferentially associated with non-heterochromatic regions.

Although the foregoing invention has been described in some detail by way of illustration and example for purposes of clarity of understanding, it will be obvious that certain changes and modifications may be practiced within the scope of the appended claims. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted.

1. A blank, adaptable for use in modifying the curvature of a patient's live cornea, comprising:
a first surface adaptable for placement directly on a surface of the patient's live cornea, and being conformable with the surface of the patients live cornea; and
at least one of the following:
a first material which, when exposed to a first energy, is adapted to increase a volume of at least a portion of the blank substantially without ablation; and
a second material which, when exposed to a second energy, is adapted to decrease a volume of at least a portion of the blank substantially without ablation.

2. A blank as claimed in claim 1, further comprising:
a second surface adaptable to be exposed to laser light; and
a third material whose properties permit light having a wavelength within the visible spectrum to pass therethrough and prevent at least some of said laser light from passing therethrough.

3. A blank as claimed in claim 1, further comprising:
a second surface, on a side opposite to said first surface; and
wherein said first and second surfaces are spaced at a distance which provides a thickness at a perimeter of said blank which is sufficient to substantially prevent the laser beam from ablating the surface of the patient's live cornea located below said perimeter.

4. A blank as claimed in claim 1, wherein:
said first energy includes light within a first wavelength range.

5. A blank as claimed in claim 4, wherein:
said light is within a blue wavelength range or an ultraviolet wavelength range.

6. A blank as claimed in claim 1, wherein:
said second energy includes light within a second wavelength range.

7. A blank as claimed in claim 6, wherein:
said light includes infrared light.

8. A blank as claimed in claim 6, wherein:
said light includes continuous or pulsed laser light.

9. A blank as claimed in claim 1, wherein:
said second energy includes at least one of microwave energy, radio frequency energy and thermal energy.

10. A blank as claimed in claim 1, wherein:
said first material includes a monomer.

11. A blank as claimed in claim 1, wherein:
said second material includes a dye.

12. A method for modifying the curvature of a patient's live cornea, comprising:
placing a blank directly on a surface of the patient's live cornea, said blank being conformable with the substantially flat surface and including at least one of a first material and a second material; and
performing at least one of the following steps:
exposing said first material to a first energy to increase a volume of at least a portion of the blank without ablation; and
exposing said second material to a second energy to decree a volume of at least a portion of the blank without ablation.

13. A method as claimed in claim 12, further comprising the steps of:
separating a layer of said cornea from a remainder of said cornea to create said surface;
repositioning said layer over said blank after said placing step; and
wherein said performing step is performed after said repositioning step.

14. A method as claimed in claim 12, wherein:
said first energy includes light within a first wavelength range.

15. A method as claimed in claim 12, wherein:
said light is within a blue wavelength range or an ultraviolet wavelength range.

16. A method as claimed in claim 12, wherein:
said second energy includes light within a second wavelength range.

17. A method as claimed in claim 16, wherein:
said light includes infrared light.

18. A method as claimed in claim 16, wherein:
said light includes continuous or pulsed laser light.

19. A method as claimed in claim 12, wherein:
said second energy includes at least one of microwave energy, radio frequency energy and thermal energy.

20. A method as claimed in claim 12, wherein:
said first material includes a monomer.

21. A method as claimed in claim 12, wherein:
said second material includes a dye.

22. A method for modifying the curvature of a live cornea, comprising the steps of:
implanting a blank having a plurality of sections within said live cornea such that a shape of said blank alters a curvature of said live cornea; and
removing at least one of said sections of said blank from said live cornea, or implanting an additional section of said blank within said live cornea, to further change a curvature of said live cornea.

23. A method for modifying the curvature of a patient's live cornea, comprising:
placing a blank directly on a surface of the patient's live cornea, said blank being conformable with the surface of the patient's live cornea and including at least one of a first material and a second material; and
performing at least one of the following steps:
exposing said first material to a first energy to increase a volume of at least a portion of the blank without ablation; and
exposing said second material to a second energy to decrease a volume of at least a portion of the blank without ablation.

24. A method as claimed in claim 23, further comprising the steps of:
separating a layer of said cornea from a remainder of said cornea to create said surface of the patient's line cornea;
repositioning said layer over said blank after said placing step; and
wherein said performing step is performed after said repositioning step.

25. A method as claimed in claim 23, wherein:
said first energy includes light within a first wavelength range.

26. A method as claimed in claim 23, wherein:
said light is within a blue wavelength range or an ultraviolet wavelength range.

27. A method as claimed in claim 23, wherein:
said second energy includes light within a second wavelength range.

28. A method as claimed in claim 27, wherein:
said light includes infrared light.

29. A method as claimed in claim 27, wherein:
said light includes continuous or pulsed laser light.

30. A method as claimed in claim 23, wherein:
said second energy includes at least one of microwave energy, radio frequency energy and thermal energy.

31. A method as claimed in claim 23, wherein:
said first material includes a monomer.

32. A method as claimed in claim 23, wherein:
said second material includes a dye.


SEQUENCE LISTING (6 sequences)

SEQ ID NO: 1 - protein, 9 aa, Artificial Sequence
  Description of Artificial Sequence myc-epitope tag
EQKISEEDL

SEQ ID NO: 2 - DNA, 31 bp, Artificial Sequence
  Description of Artificial Sequence PCR probe
gttagcactggtaattagctgctcaaaacag

SEQ ID NO: 3 - DNA, 24 bp, Artificial Sequence
  Description of Artificial Sequence PCR probe
aggaggggggtcatcaaaatttgc

SEQ ID NO: 4 - DNA, 5 bp, Artificial Sequence
  Description of Artificial Sequence GAGA factor binding motif
gagag

SEQ ID NO: 5 - DNA, 7 bp, Artificial Sequence
  Description of Artificial Sequence GAGA factor binding motif
gagagag

SEQ ID NO: 6 - DNA, 9 bp, Artificial Sequence
  Description of Artificial Sequence GAGA factor binding motif
tgagagagc